(19) United States
(12) Patent Application Publication
(10) Pub. No.: US 2026/0193594 A1
(43) Pub. Date: Jul. 9, 2026

(54) AGRICULTURAL COMPOSITIONS AND METHODS OF USE THEREOF

(71) Applicant: ARIZONA BOARD OF REGENTS ON BEHALF OF ARIZONA STATE UNIVERSITY, Scottsdale, AZ (US)

(72) Inventor: Ferran GARCIA-PICHEL, Phoenix, AZ (US)

(21) Appl. No.: 19/131,493

(22) PCT Filed: Nov. 20, 2023
(86) PCT No.: PCT/US2023/080575

§ 371 Date: May 20, 2025

Related U.S. Application Data
(60) Provisional application No. 63/427,679, filed on Nov. 23, 2022.

(51) Int. Cl. C12N 1/20; A01C 21/00; C12N 13/00

(52) U.S. Cl. CPC C12N 1/20; A01C 21/00; C12N 13/00

(57) ABSTRACT

This invention is directed to agricultural compositions and methods of using the same. For example, this invention is directed to agricultural compositions comprising a biocrust bacteria inoculum and methods of using the same.

This application claims priority from U.S. Provisional Application No. 63/427,679 filed on Nov. 23, 2022, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under 1449501 awarded by the National Science Foundation. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed agricultural compositions and methods of using the same. For example, this invention is directed to agricultural compositions comprising a biocrust bacteria inoculum and methods of using the same.

### BACKGROUND OF THE INVENTION

Drylands account for some 41% of the Earth's continental area, host 30% of the world's population and support the lion's share of the world's livestock. Up to twenty percent of them are currently classified as degraded or marginal. This situation can keep worsening in the face of two main stress factors. First, drylands experience the highest population growth rates of any biomes with a significant level of urban expansion; this can be due to increase in direct human impact. Second, arid lands will be disproportionately impacted even under the best-case scenarios of global warming.

### SUMMARY OF THE INVENTION

Aspects of the invention are drawn towards a biocrust bacteria inoculum, the inoculum comprising cyanobacteria, heterotrophic bacteria, or a combination thereof, wherein the inoculum inoculates a soil to generate a biocrust. In embodiments, the cyanobacteria comprise bacteria of the Microcoleaceae family or Coleofasciculaceae family. In embodiments, the bacteria of the Microcoleaceae family comprise Microcoleus vaginatus, Microcoleus vaginatus BNJ5, or a combination thereof. In embodiments, the bacteria of the Coleofasciculaceae family comprise Pycnacronema, Crassifilum, Crassifilum solicrustae BN2 or a combination thereof. In embodiments, the heterotrophic bacteria comprise mutualistic isolates.

Aspects of the invention are drawn towards a method of developing large biocrust nurseries, the method comprising: isolating bacteria from a remnant biocrust; performing a taxonomic assignment based on morphology of the isolated bacteria; generating an inoculum comprising bacteria similar in community composition and/or genetic identity to bacteria identified in the remnant biocrust; and inoculating a recipient soil under a photovoltaic (PV) installation to generate a biocrust in a target soil. In embodiments, the method can further comprise measuring a microbial parameter in a target soil. In embodiments, the microbial parameter comprises a visual survey, Chlorophyll a (Chl a) concentration, total extractable DNA concentration, number of 16S rRNA gene copies per unit area of soil, or combinations thereof. In some embodiments, the method further comprises inoculating a second target soil with the first target soil. In embodiments, the second target soil is in a natural setting or an artificial setting. In embodiments, the second target soil inoculates a third target soil. In embodiments, the inoculation of subsequent target soil(s) by the second or third target soils comprise a continuous process.

Aspects of the invention are drawn towards a biocrust nursery system comprising: a photovoltaic (PV) energy installation; the biocrust inoculum described herein; and a target soil, wherein the target soil is located beneath the PV energy installation. In embodiments, the system functions as both a solar power plant and as a biocrust nursery. In embodiments, the target soil inoculates a second target soil creating a cycling nursery system. In some embodiments, the system is continuous.

Aspects of the invention are drawn towards a method of testing soil for the presence of biocrust formation, the method comprising measuring microbial parameters indicative of a biocrust population. In embodiments, the microbial parameters comprise chlorophyll a (Chl a) concentration, total extractable DNA concentration. 16S rRNA gene copies, or a combination thereof. In embodiments, the Chl concentration comprises between about 5 mg m⁻² and about 50 mg m⁻². In some embodiments, the Chl concentration is greater than about 50 mg m⁻². In embodiments, the total extractable DNA concentration comprises between about 2 micrograms of DNA per cm² and about 12 micrograms of DNA per cm². In embodiments, the total extractable DNA concentration is greater than about 12 micrograms of DNA per cm². In embodiments, the 16S rRNA gene copies comprise between about 2×10⁸ copies per cm² and 20×10⁸ copies per cm². In some embodiments, the 16S rRNA gene copies comprise greater than 20×10⁸ copies per cm².

Other objects and advantages of this invention will become readily apparent from the ensuing description.

### BRIEF DESCRIPTION OF THE FIGURES

FIG. 1 shows experimental placement of survey and inoculation plots at the Poly Mount 2 solar farm with respect to layout of PV rows (Not at scale). Each plot is one square meter.

FIG. 2 shows Cyanobacteria community composition of natural and nursery-grown biocrusts. Each plot represents average relative proportions of 3 independent determinations for each taxon, resolved minimally at the Genus level. A similar analysis for bacteria/archaea at the Phylum level is shown in FIG. 8.

FIG. 3 shows distribution of biocrust cover (left panels) and Chlorophyll a in survey plots according to image analyses. Upper plot shows distribution of cover and Chl α in plots outside of the influence of PV rows, lower plots show distribution and CHl α inside of areas with PV rows. The cover means were significantly different with a p=1.0×10⁻⁶ (T-test, 2-sided, unequal variance) and with a p=0.017 (Mann-Whitney nonparametric), both run on angular-transformed percentage data. The respective Chl α means were significantly different with a with a p=0.014 using a T-test (2 sided, with unequal variance), while a non-parametric Mann-Whittney test was marginally significant (p=0.08).

FIG. 4 shows a schematic of average daily temperatures and cumulative rainfall at the study site. Lost T data during the 2021-2022 winter have been substituted with public records of the 1.5 km distant Mesa Gateway Airport, allowing for overlap (orange dots). Vertical lines mark sampling times.

FIG. 5 shows graphs the dynamics of recovery of biocrusts inside of the PV installation after harvesting with and without initial re-inoculation. Left: Biocrust recovery assessed by Chlorophyll a areal concentration (means per plot). Each panel represents a type of inoculum as labelled. Bars are standard deviations of 5 independent plots per treatment. Right: Relative changes between sampling times in Chl α concentration (averages per treatment) in experimental plots as a function of the intervening cumulative rainfall, binned by season.

FIG. 6 shows a graph of rates of biocrust recovery during the 2021/2022 season according to initial inoculation treatment. Based on linear regression with time of arithmetic means of Chlorophyll a areal concentration per treatment, with n=4 independent time points. Errors denote the standard error of the regression slope.

FIG. 7 shows an abstracted flow-through chart for the continuous operation of a PV installation under the crustivoltaics approach. Harvesting cycles are biennial and output yield is 18-fold of area harvested.

FIG. 8 shows plots of Bacteria and Archaea community composition of natural and nursery-grown biocrusts. Each plot represents average relative proportions of 3 independent determinations for each taxon, resolved at the Phylum level.

FIG. 9 shows plots of Microbial community composition of natural, nursery grown, and solar farm grown biocrust. Each plot represents average relative proportions of 3-5 independent determinations for each taxon. Variance among replicates was moderate, and full dataset can be found in FIGS. 13 and 14. Top plot presents composition of bacteria and archaea, resolved minimally at the Phylum level. Bottom plat is focused on the Cyanobacteria, resolved minimally at the Genus level.

FIG. 10 shows graphs of the effect of inoculum type on end-point biocrust development based on different biomass proxies. All proxies revealed significant treatment effects through ANOVA (Chl a: p=6×10⁻⁵; DNA: p=1.5×10⁻⁷; 16S rRNA gene copies: p=2×10⁻²). The corresponding non-parametric Kruskal Wallis tests yielded p=3×10⁻⁷ (16S rRNA), p=0.067 (Chl α) and p=8×10⁻⁵ (DNA). Significantly different treatments by letter are according to post-hoc Tuckey tests (p<0.05).

FIG. 11 shows a graph of the capacity of technologies for biocrust restoration based on published reports. The capacity is defined as the total area of soil inoculated in each report (excluding uninoculated controls, if present). The survey includes experiments carried out in laboratory, greenhouse, conditioned outdoor facilities, and full-fledged field conditions. Only a few studies inoculated areas larger than 100 m². The data distinguishes studies using cultured or nursery grown inoculants and those using transplant of nature, existing biocrusts.

FIG. 12 shows an exemplary phylogenetic tree of the 16S rRNA genes of Microcoleus vaginatus, as used in the determination and choice of isolates to be used as inoculum. Both field-retrieved sequences and sequences from isolates were placed in a preconstructed tree, to find the genetically closest isolate to use.

FIG. 13 shows a chart of cyanobacterial community composition of natural, nursery grown, and solar farm grown biocrusts in independent samples. Chart includes means and variance among replicates.

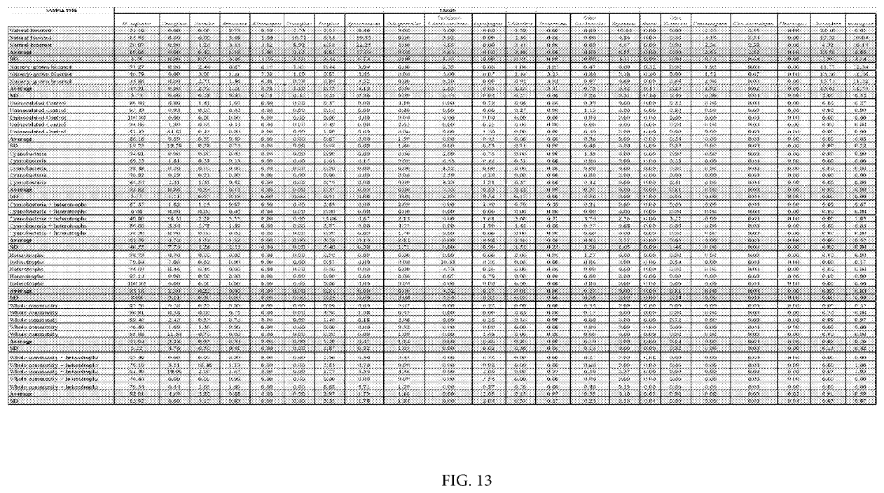

FIG. 14 shows a chart of shows a chart of bacterial and archaeal community composition of natural, nursery grown, and solar farm grown biocrusts in independent samples. Chart includes means and variance among replicates.

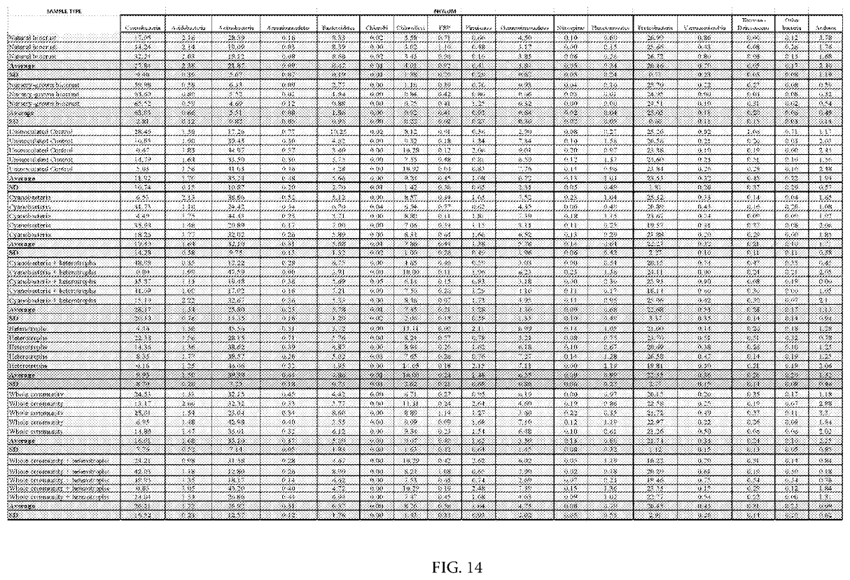

FIG. 15 shows digital photographs of biocrusts.

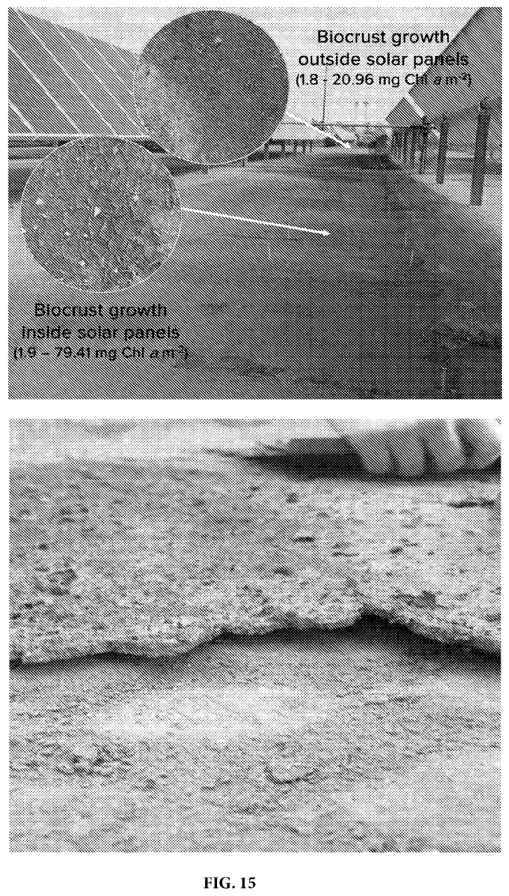

### DETAILED DESCRIPTION OF THE INVENTION

### Abbreviations and Terms

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”. “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Strategies to promote a sustainable use of arid lands and to restore what has already been lost are urgently needed. Such strategies need not only be scientifically sound and practicable, but also operate within a permissive socioeconomical framework that makes them compatible, or optimally synergistic, with current land use practices and management. Technological scale-up is perhaps the most towering, daunting impediment to effective restoration.

Aspects of the invention are drawn towards a bacteria inoculum for a biological soil crust (herein, “biocrust”). As used herein, the term “biocrust” can refer to a community of living organisms on the soil surface in arid and simi-arid ecosystems. As used herein, the term “inoculum” can refer to a composition which is used to introduce microorganisms into a substance. In embodiments, the substance can be soil, dirt, sand, clay, rocks, mud, a biocrust, or any combination thereof. For example, the substance can be a soil and the inoculum is used to inoculate the soil to generate a biocrust. In embodiments, the microorganisms in the inoculum composition can be bacteria, fungi, algae, a microorganism, or a combination thereof. In embodiments, the term “soil” can refer to a composition in which plants can grow. For example, soils can include a mixture of organic matter, minerals, gases, liquids and organisms that together support life.

In embodiments, the bacteria in the inoculum can comprise cyanobacteria, heterotrophic bacteria, or a combination thereof. As used herein, the term “cyanobacteria” can refer to aquatic, terrestrial and photosynthetic bacteria. For example, the cyanobacteria can comprise bacteria of the Microcoleaceae family or Coleofasciculaceae family. In embodiments, the bacteria of the Microcoleaceae family can comprise Microcoleus vaginatus. Microcoleus vaginatus BN15, or a combination thereof. In embodiments, the bacteria of the Coleofasciculaceae family comprise Crassifilum solicrustae BN2, Pycnacronema, Crassifilum, or a combination thereof. In some embodiments, the cyanobacteria can comprise Scytonema, Tolypothrix, or Nostoc.

As used herein, the term “heterotrophic” can refer to an organism that requires complex organic compounds of nitrogen and carbon for metabolic synthesis. In embodiments, the heterotrophic bacteria comprise mutualistic isolates. For example, the mutualistic isolates can act as bacterial probiotics. In embodiments, the heterotrophic bacteria comprise various isolates.

Aspects of the invention are directed to methods of developing large biocrust nurseries. In some embodiments, the method comprises: isolating bacteria from a remnant biocrust; performing a taxonomic assignment based on morphology of the isolated bacteria; generating an inoculum comprising bacteria similar in composition and genetic identity to bacteria identified in the remnant biocrust; and inoculating a recipient soil under a photovoltaic (PV) installation to generate a biocrust in a target soil. As used herein, the term “remnant biocrust” can refer to a biocrust that comprises a microbial composition that can be isolated to generate a target inoculum. In embodiments, the remnant biocrust is derived from nature and/or natural processes. As such, the remnant biocrust can comprise a microbial community best adapted to the site of interest. For example, the remnant biocrust can be derived from a nature.

In embodiments, isolating bacteria can refer to any process known in the art for isolating bacteria. For example, isolating can comprise growing the bacteria in media, plate streaking, colony picking, or a combination thereof. In embodiments, the the bacteria can be isolated from remnant biocrust, target soils, local soils, or any combination thereof for inoculum preparation. In embodiments, the bacteria can comprise any cyanobacterium and non-cyanobacterium. For example, the non-cyanobacteria can comprise mutualistic heterotrophs.

In embodiments, “similar” can refer to something that is done to a majority extent or degree of identity. In some embodiments, “similar” can refer to at least about 50% similar, about 55% similar, about 60% similar, about 65% similar, about 70% similar, about 75% similar about 80% similar, about 85% similar, about 90% similar, about 95% similar, about 96% similar, about 97% similar, about 98% similar, about 99% similar, about 99.5% similar, about 99.9%, or greater than 99.9%. For example, similar can refer to at least about 75%, at least about 80%, at least about 85%, at least about 90%, at least about 95%, 98%, 99%, identical.

In embodiments, communities of bacteria similar in composition to a remnant biocrust can refer to a bacterial community that has similar members in approximately similar proportions, and no major members that are not in the remnant biocrust community. In embodiments, bacteria similar in composition to a remnant biocrust can refer to a bacterial community comprised of the same Genus/Species members in similar proportions and does not contain major components absent in the remnant biocrusts.

In embodiments, bacteria similar in genetic identity to bacteria in a remnant biocrust can refer to bacteria with a high level of sequence similarity in their respective 16S rRNA gene sequence, that can be about 0.5% similar, about 1% similar, about 2.5% similar, about 5% similar, about 10% similar, about 15% similar, about 20% similar, about 25% similar, about 30% similar, about 35% similar, about 40% similar, about 45% similar, about 50% similar, about 55% similar, about 60% similar, about 65% similar, about 70% similar, about 75% similar about 80% similar, about 85% similar, about 90% similar, about 95% similar, about 96% similar, about 97% similar, about 98% similar, about 99% similar, about 99.5% similar, about 99.9%, or greater than 99.9% similar. For example, similarly in genetic identity can comprise about 90% sequence similarity for the same family. For example, similar in genetic identity can comprise about 97% to about 98% sequence similarly.

In embodiments, performing a taxonomic assignment can refer to a process of classifying and/or categorizing an organism. In embodiments, the taxonomic assignment can comprise morphological taxonomy, molecular taxonomy, or any combination thereof. In embodiments, the taxonomic assignment can comprise assignment based upon morphology, genetic sequencing, or any method known in the art. For example, the taxonomic assignment can be based upon 16S rRNA gene sequences. For example, the taxonomic assignment can be based upon the structural features of the organism.

In embodiments, generating an inoculum can refer to producing an inoculum as described herein or known in the art. As used herein, the term “inoculum” can refer to a composition comprising microbial cells and/or spores, wherein said cells/spores can propagate/germinate on or in a substrate. In embodiments, the inoculum can comprise a medium. For example, the medium can comprise a growth medium.

As used herein, the term “recipient soil” can refer to a soil that is to be inoculated by an inoculum as described herein. In embodiments, when a recipient soil is inoculated, a “target soil” is generated. As used herein, the term “target soil” can refer to a soil that is generated by compositions and/or methods described herein. For example, the target soil can be generated by inoculating a recipient soil with an inoculum. For example, a target soil can be inoculated at 5% levels of a fully developed crust. For example, if the fully developed crust has 100 mg of Chl a per square meter, the inoculation of the target soils can be around 5 mg of Chl a per square meter.

For example, a target soil can be inoculated at less than about 0.01% levels of a fully developed crust, about 0.01% levels of a fully developed crust, about 0.05% levels of a fully developed crust, about 0.10% levels of a fully developed crust, about 0.20% levels of a fully developed crust, about 0.30% levels of a fully developed crust, about 0.40% levels of a fully developed crust, about 0.50% levels of a fully developed crust, about 0.60% levels of a fully developed crust, about 0.70% levels of a fully developed crust, about 0.80% levels of a fully developed crust, about 1% levels of a fully developed crust, about 2.5% levels of a fully developed crust, about 5% levels of a fully developed crust, about 7.5% levels of a fully developed crust, about 10% levels of a fully developed crust, about 15% levels of a fully developed crust, about 20% levels of a fully developed crust, about 25% levels of a fully developed crust, about 30% levels of a fully developed crust, about 35% levels of a fully developed crust, about 40% levels of a fully developed crust, about 50% levels of a fully developed crust, about 60% levels of a fully developed crust, about 75% levels of a fully developed crust, about 80% levels of a fully developed crust, about 90% levels of a fully developed crust, about 95% levels of a fully developed crust, about 99% levels of a fully developed crust, or greater than about 99% levels of a fully developed crust. For example, the target soil can be inoculated at about 5% levels of a fully developed crust.

In embodiments, the method can further comprise measuring a microbial parameter in a target soil. As used herein, the term “microbial parameter” can refer to an analysis or test that can help determine the state, composition, and/or concentration of a microbial population. For example, the microbial parameter can comprise a visual survey, Chlorophyll a (Chl a) concentration, total extractable DNA concentration, number of 16S rRNA gene copies per unit area of soil, or combinations thereof. In some embodiments, the method further comprises inoculating a second target soil with the first target soil. In some embodiments, the second target soil is in a natural setting or an artificial setting. In some embodiments, the second target soil inoculates a third target soil. In further embodiments, the inoculation of subsequent target soil(s) by the second or third target soils comprise a continuous process.

Aspects of the invention are drawn to a biocrust nursery system comprising: a photovoltaic (PV) energy installation: a biocrust inoculum described herein; and a target soil, wherein the target soil is located beneath the PV energy installation. As used herein, the term “photovoltaic energy installation” can refer to solar panels and related equipment. In embodiments, the system functions as both a solar power plant and as a biocrust nursery. In embodiments, the target soil inoculates a second target soil creating a cycling nursery system. For example, the system is continuous.

In embodiments, the system can increase the capacity of soil restoration compared to natural rates of regeneration in similar soils not under PV installation. As used herein, the term “soil restoration capacity” can refer to the extent of target soils that can be treated from a certain restoration approach. In embodiments, the biocrust nursery system can prevent atmospheric particulate pollution compared to uncrusted soil. In embodiments, the biocrust nursery system can promote carbon sequestration compared to uncrusted soil.

A method of testing soil for the presence of biocrust formation, the method comprising measuring microbial parameters indicative of a biocrust population. In embodiments, the microbial parameters comprise chlorophyll a (Chl a) concentration, total extractable DNA concentration, 16S rRNA gene copies, or a combination thereof. In some embodiments, it can be desired to attain Chl a level that can support naturally occurring mature crusts. In embodiments, the levels can vary with soils and locations. In embodiments, the Chl concentration of the biocrusts described herein comprise between about 5 mg m⁻² and about 50 mg m⁻². For example, a Chl a concentration of above about 5 mg m⁻² can be indicative of recovery. In some embodiments, a Chl a concentration of above 20 mg m⁻² can be indicative of incipient crust. In some embodiments, a Chl a concentration above 50 mg m⁻² can be indicative of mature crusts. In embodiments, the biocrusts described herein can comprise at least about 5 mg m⁻², about 6 mg m⁻², about 7 mg m⁻², about 8 mg m⁻², about 9 mg m⁻², about 10 mg m⁻² about 11 mg m⁻², about 12 mg m⁻², about 13 mg m⁻², about 14 mg m⁻², about 15 mg m⁻², about 16 mg m⁻², about 17 mg m⁻², about 18 mg m⁻², about 19 mg m⁻², about 20 mg m⁻², about 21 mg m⁻², about 22 mg m⁻², about 23 mg m², about 24 mg m² about 25 mg m², about 26 mg m about 27 mg m⁻², about 28 mg m⁻², about 29 mg m⁻², about 30 mg m⁻², about 31 mg m⁻², about 32 mg m⁻², about 33 mg m⁻², about 34 mg m⁻², about 35 mg m⁻², about 36 mg m⁻², about 37 mg m⁻², about 38 mg m⁻², about 39 mg m⁻², about 40 mg m⁻², about 41 mg m⁻², about 42 mg m⁻² about 43 mg m⁻², about 44 mg m⁻², about 45 mg m⁻², about 46 mg m⁻², about 47 mg m⁻², about 48 mg m⁻², about 49 mg m⁻², about 50 mg m⁻², or greater than 50 mg m⁻² of Chl α.

In embodiments, the total extractable DNA concentration of the biocrusts described herein comprises between about 2 micrograms of DNA per cm² and about 12 micrograms of DNA per cm². For example, a total extractable DNA concentration of at least about 2 micrograms of DNA per cm² can be indicative of recovery. In some embodiments, a total extractable DNA concentration of at least about 5 micrograms of DNA per cm² can be indicative of incipient crust. In some embodiments, a total extractable DNA concentration above about 12 micrograms of DNA per cm² can be indicative of mature crusts. In embodiments, the biocrusts described herein can comprise at least about 3 μg cm⁻², 3.25 μg cm⁻², about 3.5 μg cm⁻², about 3.75 μg cm⁻², about 4 μg cm⁻², about 4.25 μg cm², about 4.5 μg cm⁻², about 4.75 μg cm⁻², about 5 μg cm⁻², about 5.25 μg cm², about 5.5 μg cm⁻², about 5.75 g cm⁻², about 6 μg cm⁻², about 6.25 μg cm⁻², about 6.5 μg cm⁻², 6.75 μg cm⁻², about 7 μg cm², about 7.25 μg cm², about 7.5 μg cm², about 7.75 μg cm², about 8 μg cm², about 8.25 μg cm⁻², about 8.5 μg cm⁻², about 8.75 μg cm⁻², about 9 μg cm⁻², about 9.25 μg cm⁻², about 9.5 μg cm², about 9.75 μg cm⁻² about 10 μg cm⁻², about 10.25 μg cm⁻², about 10.5 μg cm², about 10.75 μg cm², about 11 μg cm⁻², about 11.25 μg cm⁻², about 11.5 μg cm², about 11.75 μg cm⁻², about 12 μg cm², or great than about 12 μg cm⁻² of DNA.

In embodiments, the 16S rRNA gene copies of the biocrusts described herein comprise between about 2×10⁸ copies per cm² and 20×10⁸ copies per cm². For example, 16S rRNA gene copies of at least about 2×10⁸ copies per cm² can be indicative of recovery. In some embodiments, 16S rRNA gene copies of at least about 5×10⁸ copies per cm² can be indicative of incipient crust. In some embodiments. 16S rRNA gene copies of at least about 20×10⁸ copies per cm² can be indicative of mature crusts. In embodiments, the biocrusts described herein can comprise at least about 2×10⁸ copies per cm², about 2.25×10⁸ copies per cm², about 2.5×10⁸ copies per cm², about 2.75×10⁸ copies per cm², about 3×10⁸ copies per cm², about 3.25×10⁸ copies per cm², about 3.5×10⁸ copies per cm², about 3.75×10⁸ copies per cm², about 4×10⁸ copies per cm², about 4.25×10⁸ copies per cm², about 4.5×10⁸ copies per cm², about 4.75×10⁸ copies per cm², about 5×10⁸ copies per cm², about 5.25×10⁸ copies per cm², about 5.5×10⁸ copies per cm², about 5.75×10⁸ copies per cm², about 6×10⁸ copies per cm², about 6.5×10⁸ copies per cm², about 6.75×10⁸ copies per cm², about 7×10⁸ copies per cm², about 7.25×10⁸ copies per cm², about 7.5×10⁸ copies per cm², about 7.75×10⁸ copies per cm², about 8×10⁸ copies per cm², about 8.25×10⁸ copies per cm², about 8.5×10⁸ copies per cm², about 8.75×10⁸ copies per cm², about 9×10⁸ copies per cm², about 9.25×10⁸ copies per cm², about 9.5×10⁸ copies per cm², about 9.75×10⁸ copies per cm², about 10×10⁸ copies per cm², about 11×10⁸ copies per cm², about 12×10⁸ copies per cm², about 13×10⁸ copies per cm², about 14×10⁸ copies per cm², about 15×10⁸ copies per cm², about 16×10⁸ copies per cm², about 17×10⁸ copies per cm², about 18×10⁸ copies per cm², about 19×10⁸ copies per cm², about 20×10⁸ copies per cm², or greater than 20×10⁸ copies per cm² of the 16S rRNA gene.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Large portions of global arid lands are under severe, increasing anthropogenic stress, their soils already degraded or in the process of degradation. The interventional regeneration of their natural biological cover, photosynthetic communities known as biocrusts that armor the soil against erosion and fertilize it, is seen as promising for dryland restoration and sustainability. Technologies for biocrust restoration developed during the last decades are, however, invariably of high effort and low capacity, constraining application to small spatial scales. We show how existing solar power plants can be used as ad hoc biocrusts nurseries to this scaling barrier. We show experimentally that elevated solar plants indeed promote the formation of biocrust over neighboring soils, and that biocrust recovery in them is swift, for example, if re-inoculated upon harvest. We describe a mode of continuous dual operation (crustivoltaics) that is not only effective and socioeconomically attractive but can increase capacity by orders of magnitude to reach regional scales, useful in soil restoration, prevention of atmospheric particulate pollution (fugitive dust) and carbon dioxide sequestration from the atmosphere. Embodiments described herein can be used in fields where plant productivity is important, such as crop agriculture to horticulture and environmental restoration.

Drylands account for some 41% of the Earth's continental area, host 30% of the world's population and support the lion's share of the world's livestock. Up to twenty percent of them are currently classified as degraded or marginal. This situation is likely to keep worsening in the face of two main stress factors. First, drylands experience the highest population growth rates of any biomes with a significant level of urban expansion; this is likely to increase direct human impact. Second, arid lands will be disproportionately impacted even under the best-case scenarios of global warming. Strategies to promote a sustainable use of arid lands and to restore what has already been lost are urgently needed. Such strategies need not only be scientifically sound and practicable, but also operate within a permissive socioeconomical framework that makes them compatible, or optimally synergistic, with current land use practices and management. Biological soil crusts, or biocrusts, are staple communities that develop in topsoils of arid lands and other areas that can support only sparse or no plant vegetation. They rely on resident microbial (cyanobacterial, algal) or cryptogamic (lichen, moss) primary producers. Biocrusts contribute several ecosystem services to the soils they cover, notably a significant resistance against erosion through the suppression of soil loss associated with fugitive dust formation. They also act as “mantles of fertility”, a gateway for nutrients into these nutrient-poor ecosystems. Because of their beneficial effects, they are considered pivotal to dryland sustainability. Human activities such as urban sprawl, ranching, crop agriculture, military training and outdoor recreation inherently impose physical trampling on soil surfaces, and thus have deleterious effects on natural biocrust cover. Unaided recovery of biocrust is slow and can be all but halted under warming scenarios. Hence, anthropogenic pressures easily overwhelm a biocrust's ability to recover from damage. Increases in arid land population density and indirect stress exerted through global climate change have only exacerbated the problem. Currently, and as a result, large swaths of arid lands are virtually crustless, having thus been deprived of their natural suit of armor and mantle of fertility. Intervention to restore soils to their pristine state through inoculation of impacted areas with biocrusts or biocrust organisms has gained significant momentum. Early attempts using relocation of slurried, existing biocrusts indicated that harvesting, or “salvaging”, natural biocrusts is a zero net game that can be useful only under high importance and small extent restoration targets. The use of laboratory or greenhouse-based biocrust nurseries was soon attempted instead. What can have seemed originally a simple proposition, however, turned out to be more complex than thought, researchers finding a suite of unforeseen handicaps to implementation. Even refined protocols remain small-scale operations that suffice only to inoculate plots in the order of tens, at most hundreds, of square meters. Current technology can only be implemented effectively in small-area, high-importance settings, but not at the landscape scale needed for impactful ecological restoration. As an example, from our region, small particulates brought airborne by wind entrainment of soil (i.e., “fugitive dust”) constitute a secular public health hazard in metropolitan Phoenix, and studies show that local pristine desert is an insignificant contributor to fugitive dust loads. Aerosolized dust is rather sourced in the hundreds of thousands of acres of agricultural land left fallow, and still largely crustless, in Maricopa and Pinal Counties, surrounding the Phoenix metro area. Today, technological scale-up is perhaps the most towering, daunting impediment to effective restoration.

One avenue to overcome the scaling problem is to make use of existing photovoltaic (PV) energy power plants as ad hoc biocrust nurseries. Several reasons point to this. First, their elevated rows of PV panels create a milder microclimate over the soil characterized by partial shade, less extreme temperatures, and diminished evapotranspiration rates. Aside from irrigation, these are the environmental modifications consistently implemented for effective biocrust production in current protocols. More generally, such milder conditions also promote vegetation recovery under PV installations. To take advantage of the PV microclimates, the combination of crop agriculture with solar farms (the “agrivoltaic approach”) is also being pursed. In a way, solar power plants can provide a greenhouse-like setting not unlike those proven to work well in the production of man-made biocrusts. Second, solar farms can be set in agriculturally marginal lands, prominently in arid lands, and are thus available where soil restoration is needed, ready to be tapped. Third, and importantly, the sheer size of many of these alternative power generating facilities is principally sufficient to reach large scales without commensurately large investments in dedicated infrastructure, if only they can double as biocrust nurseries. Major solar projects in Arizona, for example, extend over a thousand hectares. On the other hand, a dual use would not be without benefits for the solar industry, since soil stabilization by biocrust at the large scale would prevent or diminish dust deposition on the PV panels themselves by suppressing its formation at the source. Dust deposition causes significant shading, lowering PV power and voltage outputs and requires costly operating costs for recurrent cleaning. The approach can thus be in keeping with the goal of developing sustainability solutions that offer attractive socioeconomical incentives.

In order to probe the use of PV installations in arid lands as biocrust nurseries (which we call crustivoltaics, by analogy to agrivoltaics), we can determine i) if PV installations are indeed more conducive to the natural development of biocrusts than edaphically and climatically comparable areas away from PV panel influence, ii) if harvesting of biocrusts from PV installations results in recovery within reasonably short times, and iii) if inoculation can accelerate this recovery period. For this we carried out a minimally invasive, 3-year long pilot experiment that included surveying, biocrust harvesting, inoculation and recovery monitoring in a semi-urban solar farm of the lower Sonoran Desert. Without wishing to be bound by theory, that the approach is useful and, importantly, scalable.

Visual surveys of the experimental site carried in August 2019 revealed the presence of naturally occurring biocrusts. Biocrusts cover was patently patchy across scales (from cm² to m²), and patches with conspicuous biocrust tended to be concentrated under the influence of the PV panels, while areas away from the panels had fewer patches of biocrust. Patches were made up of early-stage cyanobacterial biocrust (without dark heterocystous cyanobacteria). No lichen or moss were observed either. Developed biocrusts patches contained areal Chl a levels around 30 mg m⁻² (31 9 mg m⁻²; n=6), whereas apparently barren patches contained only traces (0.81 mg m⁻², n=6). In a follow-up sampling of patches in January, 2021 average Chl a levels in patches reached 67 5 mg m⁻², indicating that biomass development was dynamic. The soil in the site can thus naturally support the development of biocrusts, at least to a level of Chl a content between 30 and 70 mg m⁻², depending on season or antecedent conditions. This corresponds to levels seen in moderately developed cyanobacterial biocrusts of arid and semiarid natural settings. Levels of cyanocrust Chl a measured in pristine settings of the Lower Sonoran Desert, in our experience, peak maximally around 120 mg m⁻². Consistently with visual observations, the natural local crusts were dominated by cyanobacteria when analyzed using high-thruput 16S rRNA gene sequencing: more than 60% of their 16S rRNA gene reads were assignable to bundle-forming pioneer colonizers (belonging to the cyanobacterium Microcoleus vaginatus, the most common cyanobacterium in these crusts, and various genera of the family Coleofasciculaceae, such as Pycnacronema and Crassifilum). Less than 7% were attributable to heterocystous genera like Scytonema, Tolypothrix or Nostoc. This can indicate early successional biocrust in the Sonoran Desert.

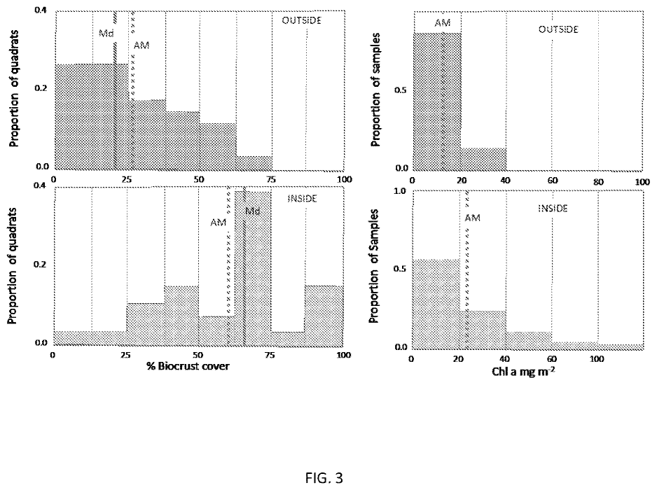

We conducted an assessment of biocrust cover in the Summer of 2022. Four preselected E-W linear transects, two under the influence of PV panels and two distant from them, were set up. Along them, 1 m² quadrats (n=14-18 per transect), one meter apart, were photographed while the soil was wet following an early morning, 10 mm-strong rain event, so that the cyanobacterial populations had migrated to the soil surface. Image analyses was used to determine the percentage area covered by biocrusts and that covered by bare soil. While plant (dead or alive) cover was minimal, it was discounted from the total area, if present. Clearly, quadrats under PV panels were conducive to the development of much more extensive biocrust, with a median of 65.4% cover (arithmetic mean of 60.2 24.1%; n=29), which is slightly above three times the 20.7% median cover attained in quadrats away from PV panels (arithmetic mean of 26.3 19.3%; n=36). These means were significantly different with a p=1.0×10−6 (T-test, 2 sided, with unequal variances) and with a p=2.3×10−6 (Mann-Whitney nonparametric) both run on angular-transformed percentage data. Over 10% of quadrats under the influence of panels were very highly colonized (>90% cover) and none were crustless. By contrast, none of the quadrats away from panels were covered above 60% and about 6% of plots had no crusts. The transects were also analyzed on the basis of Chl a areal concentration (FIG. 3). Again here, samples under the influence of PV rows contained more Chl a than those outside (23.2 23.0 vs. 12.7 6.9 mg m⁻²; n=36). The respective means were significantly different with a with a p=0.014 using a T-test (2 sided, with unequal variances), while a non-parametric Mann-Whittney test had a significance of p=0.13. We can thus show that PV installations in solar farms offer a setting naturally conducive to biocrust development in comparison to fully exposed, adjacent soil, judged both by biocrust cover and by biocrust areal biomass proxies. Cover achieved under the influence of raised PV panels tripled those found in exposed neighboring soils, Chl a concentrations almost doubled. Without wishing to be bound by theory, this is a condition sine qua non to entertain their secondary use as biocrusts nurseries. However, this represents differences in steady-states and does not inform us of the rates at which such a steady-state can have been reached.

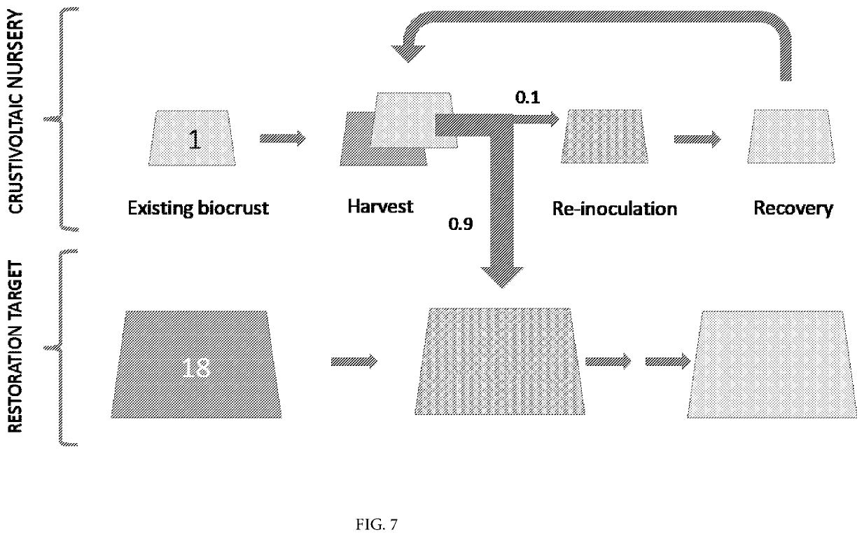

To assess rates of biocrust formation under PV panels we conducted experiments in which we excised the natural crust in small plots, and either left them untouched or provided various types of inoculants. One m² quadrats were cleared of existing biocrusts and either left to recover untreated (controls, n=5 quadrats) or inoculated with various types of inoculum (treatments, n=5 quadrats per treatment). Treatments consisted of inoculation with i) genetically pedigreed Microcoleus vaginatus BN15 and Crassifilum solicrustae BN2 (“Cyanobacteria”), ii) the same as i) but supplemented with a mixture of 5 isolates of mutualistic heterotrophs (“Cyanobacteria and heterotrophs”), iii) a polymicrobial biocrust community previously grown in an open nursery system from small amounts of natural local biocrusts (“Whole community”), iv) the same inoculum as iii) but supplemented with the mixture of heterotrophic mutualist isolates (“Whole community and heterotrophs”), and v) the mixture of heterotrophs alone (“Heterotrophs”). All plots were wetted immediately after inoculation, which took place in February 2020, so that the inoculum would benefit from the tail-end of the winter rains and be ready for summer monsoon rains. Our original intent was to monitor the plots for one year, but, unfortunately, 2020 turned out to be the driest monsoon on record for the area, popularly known as the “Non-soon”, and so we extended monitoring to the following year. Monitoring involved sacrificial, multiple sampling for biological indicators of biocrust development (Chl a concentration throughout and, additionally, DNA content and number of 16S rRNA genes at the end-point sampling). After a moderate pulse of growth following inoculation, a generalized statis or decline set in, both in controls and treatments, during the first year after inoculation, which coincided with the dry and hot spell. The second year, however, provided copious rainfall, and upward trends across controls and treatments were measurable. By the end of the second year, significant recovery had occurred in all plots, and differences according to original inoculation treatment were patent. On the basis of Chl a concentration, the treatment with the best performance had reached levels approaching the summer concentration of mature crusts in the site, although most treatments and the untreated controls fell short of this level. To gauge the general effect of rainfall on growth, we quantified the relative changes in average Chl a biomass in each plot attained between samplings. We found a moderate trend of increase in relative growth with increasing intervening cumulative rainfall (linear, R2=0.24), but the relationship between cumulative rainfall can be significantly improved if the respective periods were binned into “summer” (April, 15-October, 14) and “winter” (October 15-April 15), with R2 of 0.82 (exponential) and 0.91 (linear), respectively. In fact, unusually wet monsoons were needed to elicit net growth in the Summer, whereas much more moderate rainfall in winter had an equivalent effect on growth. Thus, winter rainfall more strongly determined biocrust development, and moderate rainfall in summer resulted in biomass loss. These results are consistent with our previous determinations of biocrust expansion in greenhouse settings and recommendations to avoid Sonoran summertime during biocrust production in nurseries. To assess treatment effects at the end point more accurately, we analyzed biocrust development using several parameters concurrently (Chl a and community DNA concentrations, as well as 16S rRNA gene copy numbers) and with increased sampling effort (n=20 per treatment). All parameters revealed significant treatment effects through ANOVA (Chl a: p=6×10⁻⁵: DNA: p=1.5×10⁻⁷; 16S rRNA gene copies: p=2×10⁻¹²). The corresponding non-parametric Kruskal Wallis tests yielded p=3×10⁻⁷ (16S rRNA), p=0.067 (Chl a) and p=8×10⁻⁵ (DNA). Tukey's multiple comparison of means test showed that the significance in difference can be attributable to a successful inoculation treatment (cyanobacteria+heterotrophs) against controls, and for some parameters also to the “cyanobacteria” treatment. To gauge the rates of recovery during the second year, we ran linear regressions of Chl a content with time using the plot Chl a means (FIG. 7). At the beginning of that year all treatments had very low biomass (0.6-1.4 mg m⁻², in the order of 1-5% of mean Chl a levels found in natural crust patches under PV installations preceding the experiment). Linear rates of recovery under this moderate weather regime ranged from 3.85 to 22.63 mg (Chl a) m⁻² yr⁻¹, depending on treatment. The cvanobacteria+heterotrophs treatment showed recovery rates that were more than fourfold those of the controls, but most were not significantly different from those in uninoculated controls.

Our control experimental plots quantified the natural recovery of biocrusts by natural processes of microbial colonization after complete harvesting. Biocrust recovery was measurable, if moderate: around 5 mg Chl a m⁻² y⁻¹ (FIG. 7) during the second year. This corresponds to a yearly recovery on the order of 15% of the long-term steady-state cover on the site. Unaided, we can expect full recovery of denuded soil under panels in some 6-8 years. However, recovery rates can be significantly accelerated if harvested areas were re-inoculated. With this effort, and in our best-performing treatments judging from their performance during the second year, one year of recovery sufficed to bring biocrust cover close to original levels. This is in spite that the inoculated microbes had to endure an unexpectedly extreme weather season during their first year that demonstrably caused significant population losses. In an unintended way, that we can still measure inoculation effects at the end of the experiment speaks for the resilience of inoculants to extreme weather and their suitability against the vagaries of climate. It is of interest to evaluate the performance of our biocrusts with respect to long term precipitation patterns. The average monsoon rainfall during the last 25 years in this area is 57 mm (CV=80%) and thus the monsoon rainfall in 2020 was indeed scant, whereas in 2021 it was on the high end. For winter rains, determinant for biocrust growth, the average is 105 mm (CV: 60%), and thus 2020/2021 was also rather meager, but 2021/2022 was very close to average. In this context, the level of biocrust recovery attained during the second year constitutes a fair long-term estimator.

Nursery-grown biocrusts matching or exceeding Chl a concentrations ever measured at the site, and still maintaining typical microbial composition, can be obtained here using 6-12 wetting/drying cycles during the production of whole community inoculum. This corresponds to absolute times of just 2-3 months. The disadvantage of the former approaches, however, is that their speed comes at the cost of intensive management, which translates into low overall capacity, making them ill-suited for implementation at large scales. By contrast, in the case of PV installations, the process management efforts are strictly constrained to harvesting and initial plot re-inoculation.

The results indicated herein point to a continuous, low-tech, low-impact operation for PV installations as sustainable sources of biocrust inoculum. In this approach one can do away with work-intensive, highly specialized nurseries by simply re-investing part of the biocrust harvest as a boost to natural recovery processes in the areas harvested. Guided by our experience at Poly Ground Mount 2, we describe how such a continuous operation can look like: for any given area of PV biocrust harvested, one can expect full recovery within 1-2 years if investing a small proportion of the harvest into its re-inoculation and without any nursery production of inoculum or other treatments. We choose 10% as a safe placeholder for this proportion, given that our initial levels at around 1-5% fell a bit short of full recovery during a normal year's growth. This still leaves 90% of the harvest to use in restoration of target degraded soils. If target soils are inoculated at 5% levels, an area equivalent to 18 times the area harvested can be treated. The original source area can then be harvested recurrently at least biennially and operated in this mode continuously.

Described herein is an illustrative assessment of PV installations for arid soil restoration using the case of Maricopa County (AZ). It sustains 191,918 ha of farmland, some 30-40% of which are left fallow because of increasing water scarcity, and constitute a problematic source of fugitive dust to the Phoenix Metro Area. The three largest PV solar stations in Maricopa County (Mesquite Solar Project. Solana Generating Station, and Arlington Valley II) cover an area of some 1623 ha (376, 777 and 469, respectively). Their dual use as biocrust nurseries to treat neighboring denuded soils at 5% inoculation level as detailed in the previous section, can principally be used to treat 29214 ha of crustless soil. This is slightly above 15% of all the agricultural land in the county, and some 45% of all its fallow land. Without wishing to be bound by theory, a biocrust regeneration time of 2 years by re-seeding at 10% inoculation, the entirety of Maricopa County fallow lands can be treated within 4-5 years. This can make a significant landscape-scale difference. An equipped, non-specialist operator can harvest 1000 m−2 meters (0.1 ha.) of PV nursery and sow 1.8 target ha per day, or some 1188 ha in a two-year period. A team of 25 such operators would suffice to conduct the entire operation in 2 years. This human resource need fits well within the common definition of a small-sized business. If even if off by an order of magnitude, one can glimpse the opportunity to make a difference. By comparison, using current nursery-based technology, and in our hands, one highly trained operator can attain maximally the seeding of a few acres per year, requiring significant technological investments in equipment, space and monitoring. The crustivoltaics approach, by doing away with the need to produce inoculum in nurseries and relying on existing, large nursery-like settings and their capacity to promote self-regeneration, thus offers cost/return benefits that are at 3-4 orders of magnitude smaller. If only at the comparative level, crustivoltaics represents a high capacity, low-cost and resource efficient new approach.

The burden of this restoration approach on PV farm operation is rather minimal, requiring only one intervention every two years, less disruptive than agrivoltaic operations. It offers the added benefit of promoting net increases in power output by reduction of dust deposition from surrounding crustless areas. Without wishing to be bound by theory, PV farm operators can be interested in crustivoltaic partnerships if the prospect of improved energy yields is part of the equation. Much more so if through such partnerships operators can take advantage of incentives for their contribution to sustainability and conservation of natural resources. One such benefit that we see as very likely is the claim of carbon sequestration credits, since biocrust are photosynthetic systems that draw down CO₂ from the atmosphere just like plants. In cyanocrusts from warm North American deserts the ratio of Carbon stock to Chl a concentration is 5 3 g C/mg (Chl a), so that an average cyanocrust with 50 mg Chl a m⁻² stocks around 0.25 Kg C m⁻² or 2.5 metric tons per ha in the top cm of soil. This general estimate is consistent with direct measurements of C stocks from cyanocrusts in China. Repopulating with biocrusts a hectare of degraded soils, will thus sequester on average 9 tons of CO₂ from the atmosphere by maturity. For comparison, this is equivalent to the cumulative sequestration of 60 pine trees during a 20 year period, according to the Winrock International calculator (winrock.org/flr-calculator/). At current carbon credit prices of $32 per ton, the additional monetization of crustivoltaics can reach in the order of $300 ha⁻¹, sufficient to cover a large portion of operating costs.

Because there are no known cases of endemism in biocrust bacteria, the unintended introduction of invasive species is virtually non-existent and the consequences of long-distance cross inoculation, without wishing to be bound by theory, are restricted to suboptimal performance or even failure. A maladaptation of inoculum to the edaphic character of target soils has been documented. Crust on gypsum-rich soils provide an extreme example, since they host a completely different set of cyanobacterial species from that found in neighboring soils with low gypsum content. In general, the presence of existing PV installations in climatically and edaphically similar settings to those of target soils can be desirable. This type of operation is what we call the cis-crustivoltaics (CCV).

Without wishing to be bound by theory, trans-crustivoltaics (TCV) can be used. In order to accommodate application to soils with different edaphic character. In TCV, a top layer of the target soil is laid down under the PV panels to obtain pre-acclimated biocrusts. This entails additional effort over CCV, and a need to seed the transplanted topsoils with an appropriate starting inoculum to circumvent slow natural inoculation mechanisms. Methods to obtain and promote the appropriate inoculum follow directly those used in our research applied to remnant natural biocrusts in the target soils.

TCV inoculum preparations are obtained by standard microbiological cultivation quality controlled by parallel DNA analyses of cultures and matching them to those existing in biocrust remnants of target soils. Motile, filamentous, bundle-forming, non-nitrogen fixing cyanobacteria, bundle specimens are picked directly from the soil surface using fine forceps under the dissecting microscope. Bundles are cleaned of soil and attached bacteria by dragging them over 2% (w/v) agar-solidified medium and then transferred into 96-well plates containing liquid medium, incubated under illumination with 20 to 30 mol (photon) m−2 s−1 under a 14 h photoperiod. Each isolate receives a strain identification code, its identity then established by DNA sequencing, and is cryopreserved until further use. To isolate heterotrophic bacteria that are mutualistic with cyanobacteria for nitrogen fixation and phosphorus solubilization cleaned bundles but incubated in nitrogen-free Burk's and NBRIY media respectively, with added organics (solidified with 1% Gellan gum) and incubated for 20, after which individual colonies were are and streaked on new plates, repeating three times for each colony transfer, to obtain pure isolates as described in detail previously. Whole community inoculum is prepared in a nursery facility under controlled conditions and rounds of recurrent irrigation/desiccation with a Fog Irrigation Soil Substrate (FISS) system, over a 1 cm deep substrate of local soil. The resulting artificial biocrusts are then collected and crumbled into mm-large pieces for manual sowing over the plot. Cyanobacterial culture inoculum was prepared as follows from the cultures obtained, pedigreed cyanobacterial strains matched genetically to those present in the natural crusts are grown under sterile conditions in the laboratory over floating paper filters, and cultures scaled up to the desired yield as explained. These cultures were then mixed with local soil and incubated once more as a mixture of culture/soil under more natural conditions in the greenhouse using the FISS technology, to “harden” the cultures. They were collected and crumbled, dried Heterotrophic mutualistic isolates are grown in the laboratory in large liquid cultures, harvested and mixed into a cocktail in equal proportions to an OD600 of 0.1. 200 mL of the cocktail was applied per m² of crusts directly to the FISS set-up or directly on the experimental plots. This inoculant materials can be prepared into dry mixtures as needed, and delivered to the target soils, after QC.

Quality control of inoculants is done through DNA sequencing and sequence analyses, matching either cultures of cyanobacteria or artificial polymicrobial biocrust to the organismal composition of remnant biocrust from the target soils, using standard methodologies from microbial community analyses based on selected marker genes. Only inoculant materials that are similar in composition and genetic identity to remnant biocrusts are then used.

Large portions of global arid lands are under severe, increasing anthropogenic stress, their soils already degraded or in the process of degradation. The interventional regeneration of their natural biological cover, photosynthetic communities known as biocrusts that armor the soil against erosion and fertilize it, is currently seen as promising for dryland restoration and sustainability. Technologies for biocrust restoration developed during the last decades are, however, invariably of high effort and low capacity, constraining application to small spatial scales. We provide a methodology for turning existing solar power plants into ad hoc biocrusts nurseries to use break this scaling barrier. We describe a mode of continuous dual operation (crustivoltaics) that is socioeconomically attractive because of its low-impact on energy operations and benefits to energy outputs, and can increase the capacity for soil restoration, prevention of atmospheric particulate pollution (fugitive dust) and promote carbon dioxide sequestration from the atmosphere capacity by orders of magnitude to reach regional scales.

In embodiments, the disclosure can be implemented with the use of existing infrastructure. In embodiments, the disclosure can scale targets increased by orders of magnitude. In embodiments, the disclosure can be a continuous, sustainable operation. In embodiments, the disclosure can allow for independence from biocrust nurseries and cultivation (in CCV mode) or only initially dependent on those (in TCV mode). Current technologies are only useful and impactful at boutique scales. Without wishing to be bound by theory, crustivoltaics can be useful and impactful at regional scales.

The interventional regeneration of the natural biological cover, photosynthetic communities known as biocrusts that armor the soil against erosion and fertilize it, is currently seen as promising for dryland restoration and sustainability in arid regions. Technologies for biocrust restoration developed during the last decades are, however, invariably of high effort and low capacity, constraining application to small spatial scales. Researchers provide a methodology for turning existing solar power plants into ad hoc biocrusts nurseries to break this scaling barrier. They describe a mode of continuous dual operation (crustivoltaics) that is socioeconomically attractive because of its low-impact on energy operations and benefits to energy outputs. These methods increase the capacity for soil restoration, prevention of atmospheric particulate pollution (fugitive dust) and promote carbon dioxide sequestration from the atmosphere capacity by orders of magnitude to reach regional scales.
More than 60% of the biocrust's 16S rRNA gene reads were assignable to bundle-forming pioneer colonizers (belonging to the cvanobacterium Microcoleus vaginatus, the most common cyanobacterium in these crusts, and various genera of the family Coleofasciculaceae, such as Pycnacronema and Crassifilum).
Less than 7% of the gene reads were attributable to theterocystous genera like Scytonema, Tolypothrix or Nostoc.
Image analyses was used to determine the percentage area covered by biocrusts and that covered by bare soil.
Treatments consisted of inoculation with i) genetically pedigreed Microcoleus vaginatus BN15 and Crassifilum solicrustae BN2 (“Cyanobacteria”),
ii) the same as i) but supplemented with a mixture of 5 isolates of mutualistic heterotrophs (“Cyanobacteria and heterotrophs”),
iii) a polymicrobial biocrust community previously grown in an open nursery system from small amounts of natural local biocrusts (“Whole community”),
iv) the same inoculum as iii) but supplemented with the mixture of heterotrophic mutualist isolates (“Whole community and heterotrophs”), and
v) the mixture of heterotrophs alone (“Heterotrophs”).
An equipped, non-specialist operator can harvest 1000 m−2 meters (0.1 ha.) of PV nursery and sow 1.8 target ha per day, or some 1188 ha in a two-year period. A team of 25 such operators would suffice to conduct the entire operation in 2 years.

### Example 2

### Methods to Restore Degraded Soils in Arid Lands at Large Scales

### Introduction to Problem and Non-Limiting, Exemplary Approach

Up to twenty percent of global drylands, which account for some 41% of the Earth's continental area, host 30% of the world's population and support the lion's share of the worlds livestock¹, are currently classified as degraded or marginal². The situation is likely to keep worsening in the face of two main stress factors. On the one hand, drylands experience the highest population growth rates of any biomes³ with a significant level of urban expansion⁴, which is likely to increase direct human impact. On the other, arid lands will be disproportionately impacted even under the best case scenarios of global warming⁵. Strategies to promote a sustainable use of arid lands and to restore what has already been lost are urgently needed. Such strategies need not only be scientifically sound and practicable, but also operate within a permissive socioeconomical framework⁶ that makes them compatible, or optimally synergistic, with current land use practices and management.

Biological soil crusts, or biocrusts, are staple communities developing in and on top soils of arid lands and other areas that can support only sparse or no plant vegetation. They rely on resident microbial (cyanobacterial, algal) or cryptogamic (lichen, moss) primary producers. Biocrusts contribute several ecosystem services to the soils they cover, notably a significant resistance against erosion⁸ through the suppression of soil loss associated with fugitive dust formation. They also act as “mantles of fertility”, a gateway for nutrients into these nutrient-poor ecosystems^{9,10}. Because of their beneficial effects, they are considered pivotal to dryland sustainability¹¹. And yet, while exhibiting remarkable resilience to the environmental extremes of temperature, desiccation, and insolation that are typical of their habitat, biocrust are brittle when dry and quite sensitive to physical compressive forces. Human activities such as urban sprawl, ranching, crop agriculture, military training and outdoor recreation inherently impose physical trampling on soil surfaces, and thus have deleterious effects on natural biocrust cover¹²⁻¹⁵. Unaided recovery of biocrust is slow^{16,17} and can be all but halted under warming scenarios¹¹, since the metabolism and growth of resident organisms is restricted to periods when the soil is wet¹⁸. In aridlands, periods of biocrust biological activity do not last much longer than the rare and short-lived events of meteoric precipitation. Hence, anthropogenic pressures can and do easily overwhelm a biocrust's ability to recover from damage. Increases in arid land population density and indirect stress exerted through global climate change have only exacerbated the problem. Currently, and as a result, large swaths of arid lands are virtually crustless, having thus been deprived of their natural suit of armor and mantle of fertility.

Intervention to restore soils to their pristine state through inoculation of impacted areas with biocrusts or biocrust organisms, long entertained as a solution^{17,19} has gained significant momentum during the last decade: a large number of independent efforts by various research groups and funding agencies to attain this goal are currently underway in arid lands the world over²⁰⁻²⁴. Early attempts using relocation of slurried, existing biocrust demonstrated that the approach can be carried out. However, harvesting, or more euphemistically “salvaging”, natural biocrusts is a zero net game that can be useful only under certain circumstances. The use of laboratory or greenhouse-based biocrust nurseries was soon attempted instead. What can have seemed originally a simple proposition, however, turned out to be more complex than thought, researchers finding a suite of unforeseen handicaps to implementation²⁴⁻²⁶. Many incremental improvements have been presented, ranging from techniques for isolation and genetic pedigreeing of isolates to match locally extant biodiversity, improved techniques for growth and monitoring of inoculants under semi-natural but controlled conditions²⁷⁻²⁹, for the avoidance of biocrust pests³⁰, for selection of optimal inoculation season^{31,32}, for enhancement of inoculum survival through hardening or soil conditioning^{32,33}, or for yield improvement through provision of so-called biocrust probiotics³⁴. Together, they have advanced this nascent field of “biocrust horticulture” to the point that one can produce high quality biocrust that will stand very good chances of surviving and further developing in the field, both for cyanobacterial biocrusts and for moss biocrusts³⁵.

However, even refined protocols remain small-scale operations that suffice only to inoculate plots tens of square meters in size. The current technology can only be implemented effectively in small-area, high-importance settings, but not at the landscape scale that is needed for impactful ecological restoration. As an example from our region, small particulates brought airborne by wind entrainment of soil (i.e., “fugitive dust”) constitute a secular public health hazard in metropolitan Phoenix³⁶, and studies show that local pristine desert is an insignificant contributor to fugitive dust loads³⁷. Aerosolized dust is rather sourced³⁸ in the hundreds of thousands of acres of agricultural land left fallow, and still largely crustless, in Maricopa and Pinal Counties, surrounding the Phoenix metro area. Today, technological scale-up is perhaps the most towering, daunting impediment to effective restoration.

One avenue to overcome the scaling problem is to make use of existing photovoltaic (PV) energy generating installations as ad hoc biocrust nurseries. Several reasons point to this. First, the elevated rows of PV panels create a milder microclimate over the soil characterized by partial shade³⁹, less extreme temperatures⁴⁰, and diminished evapotranspiration rates⁴¹. Aside from irrigation, these are precisely the environmental modifications consistently implemented for effective biocrust production in most current protocols. More generally, such milder conditions also promote vegetation recovery under PV installations⁴². To take advantage of the PV microclimates, the combination of crop agriculture with solar farms (the “agrivoltaic approach”) is currently also being pursed^{43,44}. In a way, PV installations can provide a greenhouse-like setting similar to those which can work well in the production of man-made biocrusts. Second, solar farms are oftentimes set in agriculturally marginal lands, prominently in arid lands, and are thus ready for use where soil restoration is needed, ready to be tapped. Third, and importantly, the sheer size of many of these alternative power generating facilities is principally sufficient to reach large scales without equally large investments in dedicated infrastructure, if only they can double as biocrust nurseries. Major solar projects in Arizona, for example, extend over thousands of acres. On the other hand, a dual use would not be without benefits for the solar industry, since soil stabilization by biocrust at the large scale would prevent or diminish dust deposition on PV panels themselves by suppressing its formation at the source. Dust deposition causes significant shading, lowering PV power and voltage outputs⁴⁵ and requires costly operating costs for recurrent cleaning.

In order to probe the use of PV installations in arid lands as biocrust nurseries (which we call crustivoltaics, by analogy to agrivoltaics), we sought to determine i) if PV installations are indeed more conducive to the natural development of biocrusts than edaphically and climatically comparable areas away from PV panel influence, ii) if harvesting of biocrusts from PV installations results in recovery within reasonably short times, and iii) if minimal interventions can accelerate this recovery period. For this we carried out a minimally invasive, 3-year long pilot experiment that included surveying, biocrust harvesting, inoculation and recovery monitoring in a semi-urban solar farm of the lower Sonoran Desert. We provide evidence that the approach is useful and, importantly, eminently scalable.

### Materials and Methods

Site. For our experiments, we selected a small suburban PV installation, Poly Ground Mount 2, in continuous operation by Clearway Energy during the study period, and located within ASU's Polytechnic Campus, in Mesa, Arizona (33.3011, −111.6829), because of ease of access from our Mobile Nursery Facility (some 800 ft. away) and associated continuous climate records. The substrate is a coarse-loamy, mixed, calcareous, hyperthermic Typic Torrifluvents soil of the Gilman loam series, roughly 40% sand and 22% clay, slightly alkaline pH (8), containing 12% carbonates, and classified as having a wind erodibility index of 86.

Biocrust surveying. To determine natural development of biocrusts as a function of PV panel installations, we carried out sampling along four pre-determined linear transects, two under the PV installation and two along the PV panel free space. Biocrusts cover was determined by photography and image analyses in equally spaced 1 m² quadrats 46. Photographs were taken after a rain event since this reveals existing populations of cyanobacteria that migrate to the surface where present, greening the soil 47. Determination of % cover in this way is adequate to gauge major differences, and integrates cm- and dm-patchiness effectively, but suffers from low resolution, as microbial populations of low concentration can or cannot be detectable. For this reason, we complemented our surveys with sacrificial sampling to determine relevant microbial parameters of biocrusts population density (Chl a, total extractable DNA, and number of 16S rRNA gene copies per unit area of soils, see below). Chl a is largely a cyanobacteria-specific populations proxy in the absence of algae and mosses, and widely used in biocrust research, although it is not free of uncertainties as a measure of cyanobacterial biomass²⁸. Total extractable DNA in soils is a proxy for microbial biomass in the absence of plant material and while non-crusted soils also harbor microbial DNA, its concentrations in biocrust are orders of magnitude higher⁴⁸. 16S rRNA counts are specific for bacterial and archeal biomass, also used as an alternative to Chl a in biocrust research⁴⁹, though it can overestimate small-celled microbes over large ones⁵⁰.

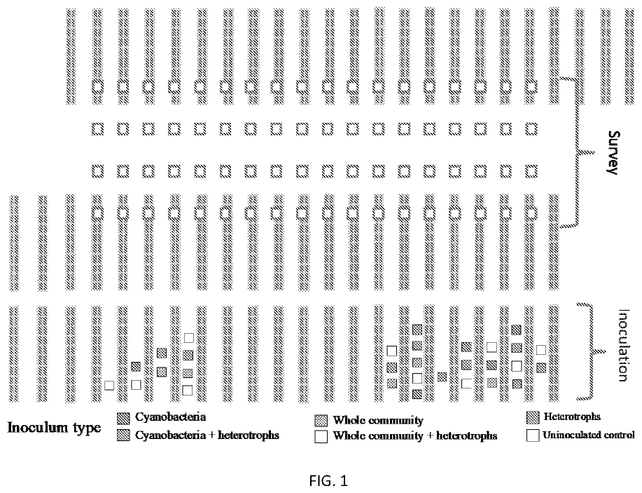

Biocrust inoculation. To determine our ability to promote growth of biocrusts under the PV nursery, we conducted interventional inoculation experiments with various treatments that lasted over 2 years. For this, a total of 30, 1 m2 plots were prepared, 5 per treatment type, placed in a randomized fashion within the area of influence of PV panel rows, avoiding placement in perimetral areas of the facility. We staked the corners of each quadrat with rebar for ease of recognition. Before inoculation, we uprooted a few forb plants present and scalped the areas of existing biocrust. No grading or perimeter walling was conducted. A spatially explicit pictorial description of the experimental design is in FIG. 1.

Isolation of local bacteria for inoculum preparation. For the isolation of motile, filamentous, bundle-forming, non-nitrogen fixing cyanobacteria, we wetted remnant biocrusts obtained locally and then picked bundles directly from the soil surface using fine forceps under the dissecting microscope. Bundles were cleaned of soil and attached bacteria by dragging them over 2% (w/v) agar-solidified Jaworski's medium and then transferred into 96-well plates containing liquid JM medium, incubated at 25±2° C. and illuminated with 20 to 30 μmol (photon) m−2 s−1 under a 14 h photoperiod 2. Once enough biomass was observed, the enrichment was inspected under the microscope, and a preliminary taxonomic assignment made on the basis of morphology. Each isolate received a strain identification code, its identity was established by DNA sequencing, and was cryopreserved until further use. To isolate heterotrophic bacteria that are mutualistic with cyanobacteria we used cleaned bundles but incubated in nitrogen-free media with added organics (solidified with 1% Gellan gum) for 20 days, after which individual colonies were picked and streaked on new nitrogen-free media plates, repeating three times for each colony transfer, to obtain pure isolates as described in detail previously (Nelson, C., Giraldo-Silva, A. & Garcia-Pichel, F. A symbiotic nutrient exchange within the cyanosphere microbiome of the biocrust cyanobacterium, Microcoleus vaginatus. The ISME Journal 15, 282-292, (2021)). Five were selected for use after testing their ability to promote the growth of cyanobacteria in culture media free of nitrogen and soluble phosphorus.

Inoculum preparation. All sources of inoculum were ultimately derived from natural biocrusts present in the surrounding area to the facility. We prepared “whole community” inoculum by cultivating these biocrust in the greenhouse of our nursery facility under controlled conditions and rounds of recurrent irrigation/desiccation with a Fog Irrigation Soil Substrate (FISS) system²⁸, over a 1 cm deep substrate of local soil. The resulting artificial biocrusts were subjected to molecular QC, then collected when they reached a Chl a concentration of 80 mg m⁻², and crumbled into mm-large pieces for manual sowing over the plot. Cyanobacterial culture inoculum was prepared as follows: two pedigreed cyanobacterial strains matched genetically to those present in the natural crusts were grown under sterile conditions in the laboratory over floating paper filters, and cultures scaled up to the desired yield as explained in detail elsewhere²⁷. These cultures were then mixed with local soil and incubated once more as a mixture of culture/soil under more natural conditions in the greenhouse using the FISS technology, to “harden’ the cultures. They were collected at a concentration of 13 mg Chla m⁻², in preparation for field inoculation. After QC, they were crumbled, dried, and mm-sized pieces of this mixture were then sown onto the experimental quadrats. Heterotrophic mutualistic isolates were grown in the laboratory in large liquid cultures, harvested and added directly to the FISS set-up or directly on the soil, as appropriate.

Chlorophyll a content. Chl a was determined spectrophotometrically after 90% acetone extraction of soil ground with mortar a pestle for 3 min²⁷. Minimally five samples of the top cm of the soil were taken randomly, covering a total area of minimally 6.8 cm², and mixed into a slurry. Slurries were placed in test tubes, volume adjusted to 10 mL with 90% acetone and extracted for 24 h at 4° C., for a single consolidated determination. Extract absorbance spectra were recorded on a Shimadzu UV-1601 spectrophotometer. Interference from scvtonemin and carotenoids was discounted using trichromatic equations²⁷.

DNA extraction and molecular methodology. Community DNA was extracted with the Powersoil Pro extraction kit (QIAGEN, Hilden, Germany) from a weighted sample of soil (about 1 g) following manufacturer's instructions and quantified using a Quant-iT™ PicoGreen® dsDNA Assay Kit (Invitrogen, Carlsbad, CA, USA). At least four of these extracts were mixed and the mixture used for quantification of total DNA, number of 16S rRNA copies and in selected sampled for determination of microbial community composition. For 16S rRNA gene quantitation, a universal (bacterial/archaeal) primer set (338F 5′-ACTCCTACGG GAGGCAGCAG-3′ (SEQ ID NO: ______), 518R 5′-GTATTACCG CGGCTGCTGG-3′ (SEQ ID NO: ______)) 52 was used. The PCR reactions were performed in triplicate using PerfeCTa SYBR Green FastMix (Quantabio, Beverly, MA, USA) in an ABI ViiA 7 thermocycler (Applied Biosystems, Foster City, CA, USA). Microbial community structure was determined through next-generation sequencing of 16S rRNA genes. The V4 region of 16S rRNA genes was amplified using general bacterial primers 515F/806R. PCR was performed in triplicate, products were pooled, and PCR protocols were performed as previously described (Gilbert, J. A. et al. Standards in Genomic Sciences 3, 249-253, (2010)). 240 ng of PCR product per sample were pooled and cleaned using the QIA Quick PCR Purification kit. DNA library concentration was quantified by qPCR using the ABI Prism® kit, brought to final concentration of 4 nM, denatured, and diluted again to a final concentration of 4 pM. 180 L of PhiX (Illumina) at a concentration of 12 pM and 150 μL of buffer HT1 (Illumina) were mixed with 270 L of the pooled library and loaded in the MiSeq Illumina sequencer, adding custom 16S rRNA sequencing primers on a paired-ends sequencing flow cell 2 (2×250 bp). Sequencing was performed in the Microbiome Analysis Laboratory at Arizona State University (Tempe, AZ, USA), yielding raw FASTQ sequence files.

Bioinformatic analyses. Paired-end reads obtained from Illumina sequencing were demultiplexed, and quality controlled using the DADA2 plugin [39] available in Qiime2 version 2020.11 [40], creating a feature table containing representative sequences (features) and their frequency of occurrence. The resulting feature table was filtered out to remove rare features. Highly variable positions were removed using MAFFT [41], and phylogenetic trees were generated using FastTree [42]. Preliminary taxonomic assignments were done with the RDP (Ribosomal Database Project) classifier [43], and representative sequences were then aligned against the Greengenes 13_8 database core reference alignment [44]. Cyanobacterial and non-cvanobacterial sequences were then split into separate feature tables [45] and the cyanobacterial features were further curated via Cvdrasil3 [46](www.cvdrasil.org).

Statistics. To assess the significance of measured parameters between two groups we used T-tests, and one-way anovas to test differences among treatments. We found that data for all biomass parameters were not normally distributed. However, one can still confidently use T-tests and ANOVAs because the number of observations was high (20 or more). To test for specific treatment drivers of significance in ANOVA tests we used Tukey's multiple comparisons of means. For % cover, data were transformed using the angular transformation for percentages before testing.

### Results

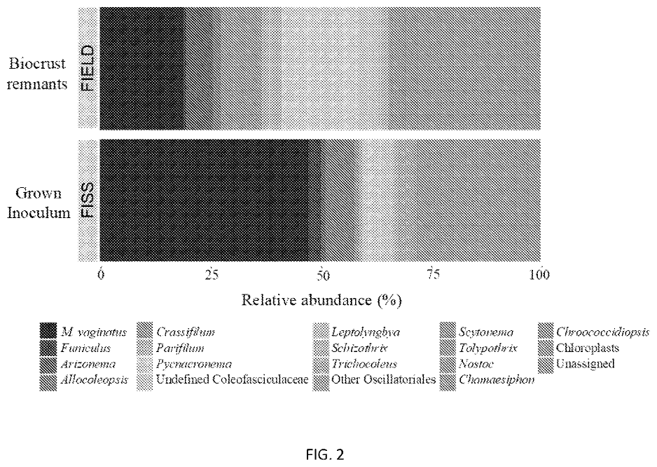

Initial visual surveys of the site carried out after a monsoon rain event in August, 2019 revealed the presence of naturally occurring biocrusts. However, biocrusts cover was patently patchy across scales (from cm² to m²), and patches with conspicuous biocrust tended to be concentrated under the influence of the PV panels, while areas away from the panels had fewer patches of biocrust. All patches were made up of early-stage cyanobacterial biocrust (without dark heterocystous cyanobacteria). No lichen or moss were observed either. Developed biocrusts patches contained areal Chl a levels around 30 mg m⁻² (31+/−9 mg m⁻²; n=6), whereas apparently barren patches contained only traces (0.8+/−1 mg m⁻², n=6). In a follow-up sampling of patches in January, 2021, after several soaking winter rain events, average Chl α levels in patches reached 67+/−5 mg m⁻², indicating that biomass development was somewhat dynamic. The soil in the site can thus naturally support the development of biocrusts, at least to a level of Chl a content between 30 and 70 mg m², depending on season or antecedent conditions. This corresponds to levels seen in moderately developed cyanobacterial biocrusts of arid and semiarid natural settings⁵⁴ Levels of cyanocrust Chl a measured in pristine settings of the Lower Sonoran Desert, in our experience, peak around 120 mg m⁻². Consistently with visual observations, the natural local crusts were dominated by cyanobacteria when analyzed using high-thruput sequencing analyses (FIG. 2): more than 60% of their 16S rRNA reads were assignable to bundle-forming pioneer colonizers (belonging to Microcoleus vaginatus, the most common cyanobacterium in these crusts, and various genera of the family Coleofasciculaceae⁵⁵, such as Pycnacronema and Crassifilum). Less than 7% were attributable to heterocystous genera like Scytonema, Tolypothrix or Nostoc. This is typical for early successional biocrust in the Sonoran Desert⁵⁶.

### Effects of PV Panels on Biocrust Cover and Density

We conducted a more thorough assessment of biocrust cover in the Summer of 2022. Four preselected E-W linear transects, two under the influence of PV panels and two distant from them were set up (FIG. 1). Along them, 18 equidistant 1 m² quadrats were photographed while the soil was wet following an early morning, 10 mm-strong rain event, so that the cyanobacterial populations had migrated conspicuously to the soil surface. Image analyses was used to determine the % area covered by biocrusts and that covered by bare soil. While plant (dead or alive) cover was minimal, it was discounted from the total area, if present. Percentage cover frequency distributions among quadrats can be found in FIG. 3. Quadrats under PV panels were conducive to the development of much more extensive biocrust, with a median of 65.4% cover (arithmetic mean of 60.2+/−24.1%; n=29), which is slightly above three times the 20.7% median cover attained in quadrats away from PV panels (arithmetic mean of 26.3+/−19.3%; n=36). These means were significantly different with a p=1.6×10−4 (T-test, 2 sided, with unequal variances, run on angular-transformed percentage data). Over 10% of quadrats under the influence of panels were very highly colonized (>90% cover) and none were crustless. By contrast, none of the quadrats away from panels were covered above 60% and about 6% of plots had no crusts. The transects were also analyzed on the basis of Chl a areal concentration. Again here, samples under the influence of PV rows contained more Chl a than those outside (23.2+/−23.0 vs. 12.7+/−6.9 mg m²; n=36). The distributions of Chl a were not normal but resembling a beta distribution with alpha of one, i.e., a decreasing distribution with a long tail towards higher values. Differences based on the steepness of decay and overall extent of this tail, that is the presence of many more patches of well-developed crust inside the influence of the PV rows (FIG. 3), as we had found for percentage cover. The respective means were significantly different with a with a p=0.014 using a T-test (2 sided, with unequal variances).

### Inoculum Preparation and Quality Control

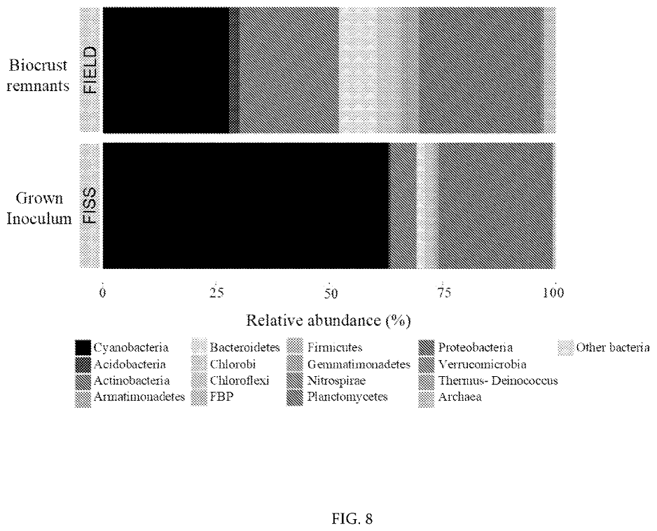

Seven strains of pioneer crust-forming cyanobacteria were obtained in culture and their 16S rRNA sequences obtained by Sanger sequencing²⁷. Comparisons with the sequences obtained from the original natural biocrust indicated that strain Microcoleus vaginatus BN15 matched perfectly (100%) the most abundant cyanobacterial sequence variant found in the field samples and was chosen for inoculation (FIG. 8). Since we obtained no cultures representative of the second most common field sequence variant (assignable to Pycnacronema), we also used strain Crassifilum solicrustae BN15, which matched 99.6% the third most abundant sequence variant in field samples. Both strains grew well when inoculated in the scale-up in-soil nursery settings run under FISS approaches²⁸ to a concentration of 13 mg Chl a m⁻², and the products were mixed in equal proportion and used to inoculate the corresponding experimental “cyanobacteria” plots at a final concentration of 1.36 mg Chl a m⁻². Thirty strains of heterotrophic cyanosphere isolates were obtained, and those were checked in culture for their ability to promote growth of the chosen cyanobacterial strains in media lacking dissolved N and dissolved P (i.e., their mutualistic potential) according to test previously described⁵¹. Five positive isolates were selected, grown in the laboratory, mixed in equal proportions, and used as biocrust probiotics, being added either directly on the appropriate plots (treatment: heterotrophs) or to the final stage of nursery growth (treatments: cyanobacteria+heterotrophs and whole community+heterotrophs) directly from liquid suspensions. Whole community inoculum was also grown under the FISS approach in the nursery with initial inoculation using small amounts of natural crust, to a concentration of 80 mg Chl a m⁻². This mixed biocrust community was also characterized genetically, to ensure that the composition contained biocrust pioneers genetically close to the ones existing in the natural crust, which was indeed the case (FIG. 2). It was used to inoculate “whole community” treatments at a final in-plot concentration of 3.52 g m².

Recovery after Biocrust Harvest

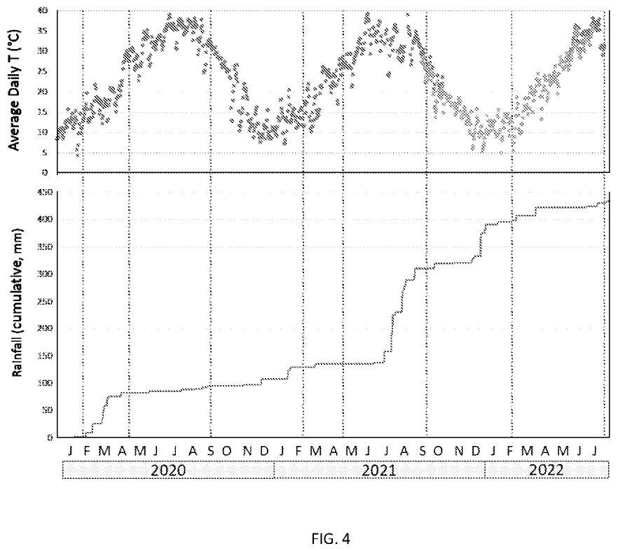

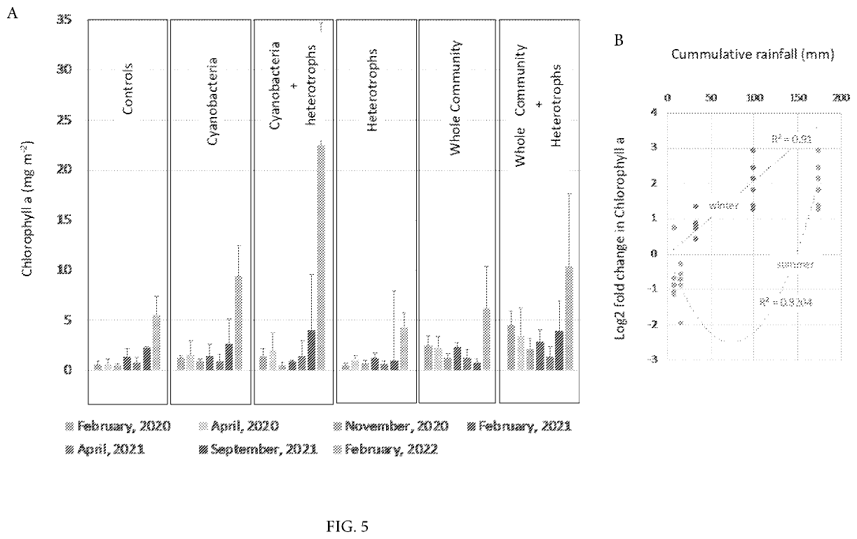

We set up an experiment to test recovery of biocrusts under the influence of PV panels with and without intervention, in which 1 m² quadrats were cleared of existing biocrusts and either left to recover untreated (controls, n=5 quadrats) or inoculated with various types of inoculum (treatments, n=5 quadrats per treatment). Treatments consisted of inoculation with i) genetically pedigreed Microcoleus vaginatus BN15 and Crassifilum solicrustae BN2 (“Cyanobacteria”), ii) the same as i) but supplemented with a mixture of 5 isolates of mutualistic heterotrophs (“Cyanobacteria and heterotrophs”), iii) a polymicrobial biocrust community previously grown in an open nursery system from small amounts of natural local biocrusts (“Whole community”), iv) the same inoculum as iii) but supplemented with the mixture of heterotrophic mutualist isolates (“Whole community and heterotrophs”), and v) the mixture of heterotrophs alone (“Heterotrophs”). All plots were wetted once after inoculation. Inoculation took place in February 2020, so that the inoculum would benefit from the tail-end of the winter rains and be ready for summer monsoon rains. Our original intent was to monitor the plots for one year, but, unfortunately, 2020 turned out to be the driest monsoon on record for the area (FIG. 4), popularly known as the “Non-soon”, and so we extended monitoring to the following year. Monitoring involved sacrificial, multiple sampling for biological indicators of biocrust development (Chl a concentration, throughout and additionally, DNA content and number of 16S rRNA genes at the end-point sampling). The dynamics of recovery based on Chl a are presented in FIG. 5.

After a moderate growth spurt following inoculation, a generalized statis or decline both in controls and treatments set in during the first year after inoculation, which coincided with the extremely dry and hot spell. The second year received much stronger rainfall, and upward trends across controls and treatments were measurable. By the end of the second year, significant recovery had occurred in all plots, and differences according to original inoculation treatment were patent (FIG. 5 left). On the basis of Chl a concentration, the treatment with the best performance (cyanobacteria+heterotrophs) had reached levels approaching the summer concentration of mature crusts at the site, although most treatments and the untreated controls fell short. To gauge the general effect of rainfall on growth, we quantified the number of doublings (fold change) in average Chla biomass in each plot attained between samplings (FIG. 3 right). We found a moderate trend of increase in relative growth with increasing intervening cumulative rainfall (linear fit, R²=0.24), but the relationships improved if the respective periods were binned into “summer” (April, 15-November, 14) and “winter” (November 15-April 15) seasons, with R² of 0.8 and 0.91, respectively (FIG. 3B). In fact, an unusually wet monsoon in 2021 was needed to elicit significant net growth, whereas winter rainfall reached similar effects at much lower rainfall. Moderate rainfall in summer resulted in biomass loss. Thus, winter rainfall more strongly determined biocrust development overall. This is likely because under extreme T in summer, evaporation is very effective, shortening the active times of microorganisms, overwhelming their capacity to transition between active growth and dormancy⁵⁷. These results are consistent with our previous determinations of biocrust expansion in greenhouse settings and with recommendations to avoid summertime during biocrust production in nurseries of the Sonoran³¹.

To more accurately assess treatment effects at the end point, we analyzed biocrust development using several parameters concurrently (Chl a and community DNA concentrations) and with increased sampling effort (n=20 per treatment). ANOVA and non-parametric Kruskal Walllis test for Chl a concentrations found treatment effects to be significant with p=6.7×10⁻⁵ and p=0.06, respectively. For DNA concentration, ANOVA found p=1.5×10⁻⁷ and Kruskal Wallis p=9×10⁻⁶. Tukey's, multiple comparison of means test showed that the significance in difference was attributable exclusively to the most successful inoculation treatment (cyanobacteria+heterotrophs) against all others, regardless of parameter considered.

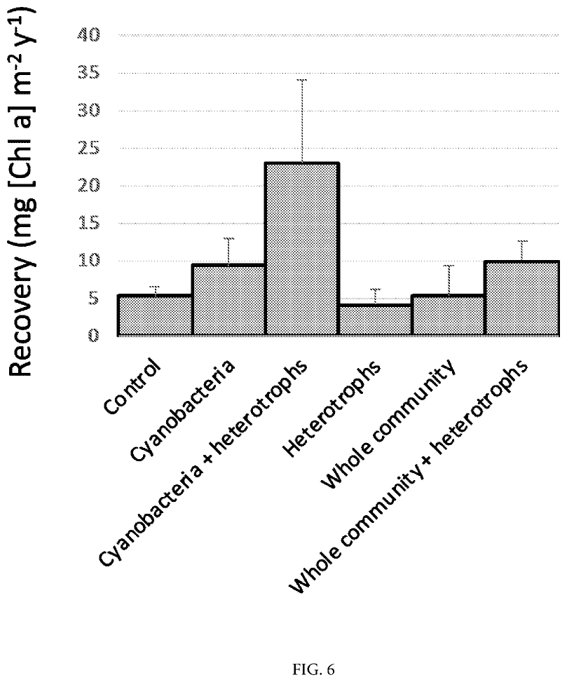

To gauge the rates of recovery during the second year, we ran linear regressions of Chl a content with time using the plot Chl a means (FIG. 6). At the beginning of that year all treatments had very low biomass (0.6-1.4 mg m⁻², in the order of 1-5% of mean Chl a levels found in natural crusts under PV installations preceding the experiment). Linear rates of recovery under moderate weather regimes ranged from 3.85 to 22.63 mg (Chl a) m⁻² yr−1, depending on treatment. Several of the inoculation treatments showed recovery rates that were more than double those of the controls, while most were not much different from those in uninoculated controls.

### Discussion

### PV Installations as Biocrust Nurseries.

We can show that PV installations in solar farms offer a setting naturally conducive to biocrust development in comparison to fully exposed, adjacent soil, judged both by biocrust cover and by biocrust areal biomass proxies (FIG. 3). Cover and areal biomass achieved under the influence of raised PV panels at least doubled those found in exposed neighboring soils. Without wishing to be bound by theory, this is a condition sine qua non to entertain their secondary use as biocrusts nurseries. However, this represents differences in steady-states and does not necessarily inform us of the rates at which such a steady-state can have been reached.

Our control experimental plots quantified the natural recovery of biocrusts by natural processes of microbial colonization after complete harvesting. Given the low remaining levels of cyanobacteria in undercrust soil after harvesting, natural inoculum sources for these plots must have been either aeolian (through deposition) or by overland water flow. Because there were no rain events large enough to elicit significant overland flow during the study period, the former mechanism was likely the most significant at play. Consistently, wet deposition of bacteria from the “aerobiome” can provide up to 108 bacterial 16S rRNA gene copies cm⁻² month-1 during rainy seasons⁵⁸. In any event, biocrust recovery was measurable, if moderate: around 5 mg Chl a m−2 y−1 (FIG. 3) during a year of average precipitation. This corresponds to a yearly recovery on the order of 15% of the long-term steady-state cover on the site. Without wishing to be bound by theory, unaided, one can achieve full recovery of denuded soil under panels in some 6-8 years.

However, without wishing to be bound by theory, recovery rates can be accelerated if harvested areas were re-inoculated. With this effort, and in our best-performing treatments (FIG. 4, 6) judging from their performance during the second year, one year of recovery sufficed to bring biocrust cover close to original levels. This is in spite that the inoculated microbes had to endure an unexpectedly extreme weather season during their first year in the soil that demonstrably caused significant population loses. Surprisingly, we can still measure inoculation effects at the end of the experiment speaks for the resilience of inoculants to extreme weather and their suitability against the vagaries of climate.

The disadvantage of the previous approaches, is that their speed comes at the cost of intensive management, which translates into low overall capacity, making them ill-suited for implementation at large scales. By contrast, in the case of PV installations, the process management efforts are strictly constrained to harvesting and initial plot re-inoculation.

### PV Installations as a Sustainable Resource for Biocrust Inoculum

The results obtained in this work indicate a continuous, low-tech, low-impact operation for PV installations as sustainable sources of biocrust inoculum. In this approach one can do away with work-intensive, highly specialized nurseries by simply re-investing part of the biocrust harvest as a boost to natural recovery processes in the areas harvested. Guided by our experience at Poly Ground Mount 2, we describe in FIG. 7 what such a continuous operation can look like: for any given area of PV biocrust harvested, one can expect full recovery within 1-2 years if investing a small proportion of the harvest into its re-inoculation and without any nursery production of inoculum or other treatments. We chose 10% as a safe placeholder for this proportion, given that our initial levels at around 1-5% fell a bit short of full recovery during a normal year's growth. This still leaves 90% of the harvest to use in restoration of target degraded soils. If target soils are inoculated at 5% levels, an area equivalent to 18 times the area harvested can be treated. The original source area can then be harvested recurrently at least biennially and operated in this mode continuously.

### Assessing the Scalability Promise of Crustivoltaics

We provide here an illustrative assessment of PV installations for arid soil restoration using the case of Maricopa County (AZ). It sustains 191,918 ha of farmland⁵⁹, some 30-40% of which are left fallow because of increasing water scarcity, and constitute a problematic source of fugitive dust to the Phoenix Metro Area. The three largest PV solar stations in Maricopa County (Mesquite Solar Project, Solana Generating Station, and Arlington Valley II) cover an area of some 1623 ha (376, 777 and 469, respectively). Their dual use as biocrust nurseries to treat neighboring denuded soils at 5% inoculation level as detailed herein, can be used to treat 29214 ha of currently crustless soil. This is slightly above 15% of all the agricultural land in the county, and some 45% of all its fallow land. Assuming a biocrust regeneration time of 2 years by re-seeding at 10% inoculation, all fallow lands can be treated within 4-5 years. The potential is there to make a significant landscape-scale difference. An equipped, non-specialist operator can harvest 1000 m⁻² meters (0.1 ha.) of PV nursery acre and sow 1.8 target ha per day, or some 1188 ha in a two-year period. A team of 25 such operators can suffice to conduct the entire operation in 2 years. This human resource need fits well within the common definition of a small-sized business. By comparison, using nursery-based technology, and in our hands, one highly trained operator can attain maximally the seeding of a few acres per year, requiring significant technological investments in equipment, space and monitoring. The crustivoltaics approach, by doing away with the need to produce inoculum in nurseries and relying on existing, large nursery-like settings and their capacity to promote self-regeneration, thus offers cost/return benefits that are at 3-4 orders of magnitude smaller. If only at the comparative level, crustivoltaics represents a high capacity, low-cost and resource efficient new approach.

### a Win-Win Situation

The burden of this restoration approach on PV farm operation is rather minimal, requiring only one minimally invasive intervention every two years, certainly much less disruptive than agrivoltaic operations. It offers the added benefit of promoting net increases in electrical output by reduction of dust deposition from surrounding crustless areas. Without wishing to be bound by theory, PV farm operators can be interested in crustivoltaic partnerships if the prospects of improved energy yields are part of the equation. Much more so if through such partnerships the companies can take advantage of incentives for their contribution to sustainability and conservation of natural resources.

Without wishing to be bound by theory, one such benefit can be carbon sequestration credits, since biocrust are photosynthetic systems that draw down CO₂ from the atmosphere just like plants. The credits can be significant. In cyanocrusts from warm North American deserts, the ratio of Carbon stock to Chl a concentration is 5+/−3 g C/mg (Chl a), calculated from data (n=15) in references^{9,46}, so that an average cyanocrust with 50 mg Chl a m⁻² stocks around 0.25 Kg C m⁻² or 2.5 metric tons per ha in the top cm of soil. This general estimate is consistent with direct measurements of C stocks from cyanocrusts in China 60. Repopulating with biocrusts a hectare of degraded soils, will thus sequester on average 9 tons of CO₂ from the atmosphere by maturity. For comparison, this is equivalent to the cumulative sequestration of 60 pine trees during a 20 year period, according to the Winrock International calculator (winrock.org/fir-calculator/) based on ref.⁶¹. At current carbon credit prices of $32 per ton, the additional monetization of crustivoltaics can reach in the order of $300 ha⁻¹, sufficient to cover a large portion of operation costs.

### Constraints

Crustivoltaics as explained here in simple terms can face some predictable constraints or limitations. Because there are no known cases of endemism in biocrust bacteria, the unintended introduction of invasive species is virtually non-existent and the consequences of long-distance cross inoculation are likely restricted to suboptimal performance or even failure. A similar maladaptation of inoculum to the edaphic character of target soils has been documented⁶⁷. Crust on gypsiferous soils, provide an extreme example, since they host a completely different set of cyanobacterial species from that found in neighboring soils with low gypsum content^{64,65}. A certain degree of match between soil under PV panels and target soil can be recommendable. Of course, one can provide a top layer of target soil under the PV nurseries, but this can entail additional effort and the need to seed it with an appropriate starting inoculum to circumvent slow aeolian processes. In general, the presence of existing PV installations in climatically and optimally edaphically settings similar to those of target soils can be desirable.

### Example 3

### Dual Use of Solar Power Plants as Biocrust Nurseries for Large-Scale Arid Soil Restoration

Large portions of global arid lands are under severe, increasing anthropogenic stress, their soils progressively degrading or already degraded. The interventional regeneration of the natural cover of these soils—photosynthetic communities known as biocrusts that armour them against erosion and fertilize them—is regarded as promising for dryland restoration and sustainability. Technologies for biocrust restoration developed during the past decades are, however, invariably of high effort and low capacity, constraining application to small spatial scales. We tested the notion that crustivoltaics, where solar power plants are used as ad hoc biocrusts nurseries, can break this scaling barrier. We show experimentally that solar plants indeed promote the formation of biocrust over neighbouring soils, doubling biocrust biomass and tripling biocrust cover, and that after biocrust harvesting, recovery is swift particularly if re-inoculated. Our results point to a mode of continuous dual operation that is not only effective and socioeconomically attractive but can also increase capacity by orders of magnitude to reach regional scales.

Drylands account for some 41% of the Earth's continental area, host 30% of the world's population and support a major share of the world's livestock¹. Up to 20% of drylands are currently classified as degraded or marginal². This situation is likely to worsen as drylands experience high population growth rates³, expansion⁴ and disproportionately strong impacts of global warming⁵. Strategies for future dryland sustainability and restoration of what has already been lost are thus urgently needed. In promoting sustainable use and helping curb its ongoing degradation, strategies are needed that are not only scientifically sound and practicable, but also operate within a permissive socioeconomic frame-work⁶ that makes them compatible, even synergistic, with current management practices.

Biological soil crusts, or biocrusts, are staple communities of arid and other soils that support only sparse or no plant vegetation. Based on microbial (cyanobacterial, algal) or cryptogamic (lichen, moss) primary producers, they contribute resistance against erosion⁷, sup-pressing soil loss as fugitive dust, and act as ‘mantles of fertility’, fixing carbon dioxide as organic carbon into the soil and providing nutrients into these nutrient-poor ecosystems^{8,9}. Hence, they are considered pivotal to dryland sustainability¹⁰. However, human activities such as urban sprawl, ranching, crop agriculture, military training and outdoor recreation inherently impose physical trampling on soil surfaces, which is deleterious to biocrusts^{11,12}. Biocrust recovery can take anywhere from years to decades, depending on the intensity of disturbance^{13,14} since growth is restricted to periods when the soil is wet¹⁵. Anthropo-genic pressures thus easily overwhelm a biocrust's ability to recover from damage. Currently, and as a result, large swaths of drylands are virtually crustless, deprived of their natural suit of armour and mantle of fertility. Dryland population growth and global climate change will only exacerbate the problem. In the Phoenix Metro area, for example, pristine desert does not contribute much to fugitive dust load¹⁶, but large expanses of fallow and crustless agricultural land do¹⁷. This fugitive dust from local soils constitutes a secular public health hazard in metropolitan Phoenix¹⁸.

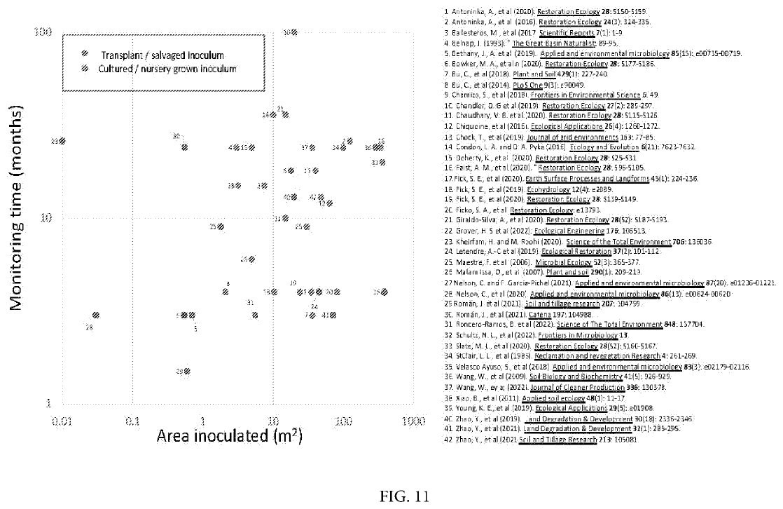

Interventional soil restoration through inoculation of impacted areas with biocrusts or biocrust organisms, has long been entertained^{15,19,29}, has notably gained momentum during the past decade; many independent efforts worldwide are currently underway²¹⁻²³. Early attempts that used relocation of existing biocrust demonstrated that the approach was feasible. However, harvesting, or euphemistically ‘salvaging’, natural biocrusts can be useful only under high importance and small extent restoration targets. The use of laboratory or greenhouse-based biocrust nurseries was soon attempted instead. This was more complex than anticipated, researchers finding a suite of unforeseen handicaps to implementation. Laboratory-based nurseries require efforts in cultivation, pedigreeing, scaling-up and acclimation of inoculum, while greenhouse-based nurseries require time-consuming optimization of incubation conditions and monitoring of microbial composition, but cannot provide inocula of precisely tailored composition^{21,22,24}. While many incremental improvements have been presented²⁴⁻²⁷ even refined protocols today remain small-scale, deployed within footprints smaller than hundreds of square metres (FIG. 11). Current technology simply cannot be implemented at the landscape scale needed for impactful ecological restoration without multiplying our current technology, facilities and efforts by orders of magnitude. Today, technological scale-up is perhaps the most towering and daunting impediment to effective restoration.

For several reasons, we contend that one avenue to overcome the scaling problem is to make use of existing photovoltaic (PV) energy power plants as ad hoc biocrust nurseries. First, elevated rows of PV panels create a microclimate over the soil characterized by partial shade²⁸, less extreme temperatures²⁹ and diminished evapotranspiration rates³⁰. Such milder conditions also promote vegetation recovery under PV installations³¹, which has led to the combination of crop agriculture with solar farms to pursue the ‘agrivoltaic approach’^{32,33}. Solar farms can thus act as a greenhouse-like setting similar to those that work well in the production of human-made biocrusts. Second, solar farms are oftentimes set in agriculturally marginal lands, prominently in drylands, and are thus available where soil restoration is needed. Third, and importantly, the sheer size of many of these installations can suffice to reach large scales without commensurately large investments in dedicated infrastructure. A dual use can also benefit the solar industry since biocrust-mediated soil stabilization at the large scale can prevent or diminish dust deposition on the PV panels themselves, which can decrease their power and voltage outputs³³. Finally, its dual use can help justify the construction of plants in natural areas, as there can be a net benefit to the natural environment. All these can thus fit the goal of developing sustainability solutions that offer attractive socioeconomic incentives.

To investigate the PV installations as biocrust nurseries (which we call ‘crustivoltaics’, by analogy to agrivoltaics), we can determine whether (1) PV installations are more conducive to the development of biocrusts than edaphically and climatically comparable areas away from PV panel influence, (2) harvesting of biocrusts from PV installations results in recovery within reasonably short times and (3) inoculation can accelerate this recovery period. For this we carried out a 3-year-long experiment that included surveying, biocrust harvesting, inoculation and recovery monitoring in a semi-urban solar farm of the lower Sonoran Desert. We provide clear evidence that the approach is feasible, useful and, importantly, eminently scalable.

### Results

Visual inspection of the experimental site (Poly Mount 2 Solar Plant in Mesa, Arizona; FIG. 1) after a monsoon rain event in August 2019 revealed the presence of natural biocrusts, which were patently patchy across scales (from cm² to m²). These were cyanobacterial biocrust without substantial populations of dark heterocystous cyanobacteria, lichen or moss. Developed biocrust patches contained areal chlorophyll a (Chl a) levels around 30 mg m⁻² (31±9: n=6), whereas apparently barren patches had only traces (0.8±1 mg m⁻², n=6). In January 2021, after soaking winter rains totaling 175 mm (a value that is high but not extreme compared with long-term means), average Chl a in patches reached 67±5 mg m⁻², indicating that biomass was dynamic. The site can thus naturally support biocrust to a level between 30 and 70 mg Chl a m⁻², depending on season or antecedent conditions.

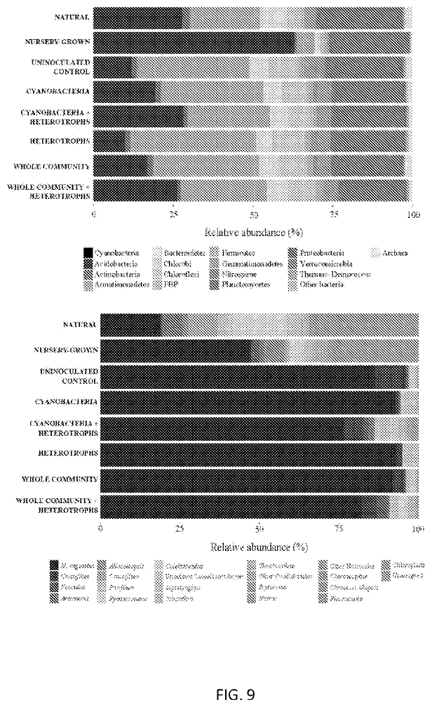

This corresponds to moderately developed cyanobacterial biocrusts of arid and semiarid natural settings³⁴. Cyanocrusts in the Lower Sonoran peak around 120 mg Chl a m⁻² Consistently with visual inspection, the natural local crusts were dominated by cyano-bacteria when analyzed using 16S ribosomal RNA gene sequencing (FIG. 9): more than 60% of their 16S rRNA gene reads were assignable to bundle-forming pioneer colonizers (Microcoleus vaginatus, the most common cyanobacterium in biocrusts, and various genera of the family Coleofasciculaceae³⁵). Less than 7% were attributable to heterocystous genera, as is typical for early successional biocrust in the Sonoran Desert³⁶.

### Effects of PV Panels on Biocrust Cover and Density

We conducted a thorough assessment of biocrust cover in the Summer of 2022. Four preselected east-west transects, two under the influence of PV panels and two distant from them, were analyzed (FIG. 1). Along them, 1 m² quadrats 1 m apart were photographed while the soil was wet following a rain event, so that cyanobacteria had migrated conspicuously to the soil surface³⁷. The percentage biocrust a real cover was determined by image analyses performed using the open-source ImageJ software. FIG. 3 shows the resulting cover frequency distributions. Quadrats under PV panels contained significantly more extensive biocrusts (median: 65.4% cover, arithmetic mean: 60.2±24.1%; n=29), slightly above three times the 20.7% median attained in quadrats away from PV panels (arithmetic mean: 26.3 19.3%; n=36). Over 10% of quadrats under the influence of panels were very highly colonized (>90% cover) and none were crustless. No quadrats away from panels were covered above 60% and about 6% had no crusts. The transects were also analyzed by Chl a areal concentration (FIG. 3). Again here, Chl a was higher inside than outside the influence of PV rows (23.2±23.0 vs 12.7±6.9 mg m²; n=36).

### Inoculum Preparation and Quality Control

From the seven strains of cyanobacteria cultivated, we selected two for field inoculation: Microcoleus vaginatus BN15 (whose 16S rRNA gene matched 100% the most abundant cyanobacterial sequence variant found in the field) and Crassifilum solicrustae BN27, which matched the third most abundant field sequence with 99.6% sequence similarity. We obtained no cultured representative of the second most common field sequence variant (Pycnacronema). Both strains were inoculated into nursery settings run under FISS (fog irrigated soil substrate system²⁷) and grown over 14 wet-dry cycles for 42 d to a final biomass of 13 mg Chl a m⁻², mixed in equal proportions and then inoculated in the corresponding experimental ‘cyanobacteria’ plots (FIG. 1) at a final concentration of 1.36 mg Chl a m⁻². All seven strains of heterotrophic mutualistic isolates obtained were grown and added to the appropriate plots from liquid suspensions. Whole community inoculum was grown from small amounts of natural crust to a concentration of 80 mg Chl a m⁻² under the FISS approach (14 wet-dry cycles, 42 d). The resulting community was also characterized genetically, ensuring that it contained typical biocrust pioneers genetically close to the ones existing naturally (FIG. 9), and inoculated in experimental plots at 3.52 g m².

Recovery after Biocrust Harvest

We set up an experiment to test the potential for biocrust recovery under the influence of PV panels with and without intervention, in which 1 m² quadrats were cleared of existing biocrusts and either left to recover untreated (controls, n=5) or inoculated with various inoculum types (treatments, n=5 quadrats per treatment, FIG. 1). Inoculum types were: (1) Microcoleus vaginatus BN15 and Crassifilum solicrustae BN27 (‘Cyanobacteria’), (2) the same as (1) but supplemented with a mixture of mutualistic heterotrophs (‘Cyanobacteria and heterotrophs’), (3) the polymicrobial biocrust previously grown in an open nursery system (‘Whole community’), (4) the same inoculum as (3) but supplemented with a mixture of heterotrophic isolates (‘Whole community and heterotrophs’) and (5) a mixture of heterotrophs alone (‘Heterotrophs’). Plots were wetted immediately after inoculation, which took place in February 2020 so that they would benefit from the tail-end of the winter rains and be ready for summer monsoon rains. Our intent was to monitor plots for 1 yr, but 2020 turned out to be the driest monsoon on record (FIG. 4), popularly known as the ‘Non-soon’, so we extended monitoring to the following year. Monitoring involved sacrificial sampling for biomass indicators (Chl a concentration throughout and, additionally, DNA content and number of 16S rRNA genes at the end-point sampling in February 2022).

The dynamics of recovery based on Chl a are shown in FIG. 5 panel A. After a moderate pulse of growth following inoculation, a generalized stasis or decline set in, both in controls and treatments, coinciding with the extremely dry and hot spell. The second year provided copious rainfall, and upward trends across controls and treatments were measurable. By the end of the second year, recovery had occurred in all plots, and differences according to original inoculation treatment were patent (FIG. 5 panel A). The best-performing treatment (cyanobacteria+heterotrophs) had reached a Chl a level approaching the summer concentration of natural crusts at the site, although most treatments and the untreated controls fell short of this. To gauge the general effect of rainfall on growth, we quantified the changes in average Chl a biomass attained between samplings (FIG. 5 panel B). We found a moderate trend of increase in relative growth with increasing intervening cumulative rainfall (linear, R²=0.24), but the relationship can be substantially improved if the respective periods were binned into ‘summer’ (15 April to 14 October) and ‘winter’ (15 October to 14 April), with R² of 0.82 (quadratic) and 0.91 (linear), respectively. This reveals that unusually wet monsoons were needed to elicit net growth, whereas much more moderate rainfall in winter had an equivalent effect on growth. Thus, winter rainfall more strongly determined biocrust development and moderate rainfall in summer resulted in biomass loss. This is consistent with previous determinations of biocrust expansion in greenhouse settings²⁶ and with recommendations to avoid Sonoran summertime during biocrust production in nurseries²⁶.

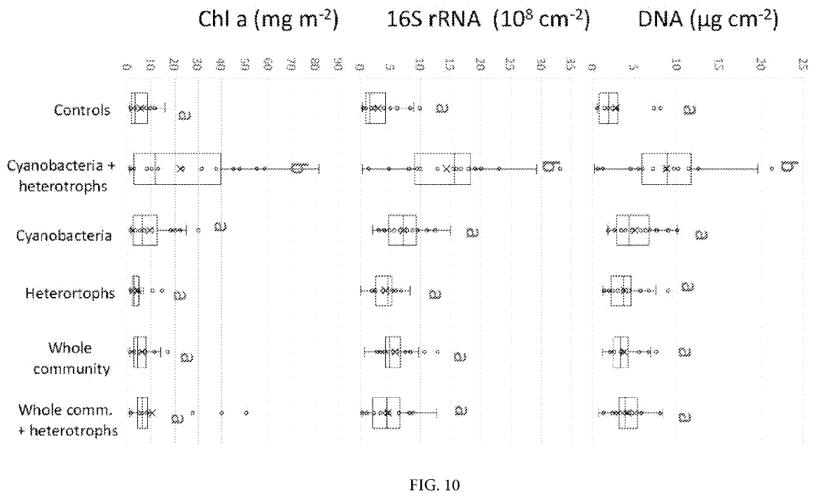

To assess treatment effects at the end point more accurately, we analyzed biocrust development using several parameters concurrently (Chl a and community DNA concentrations, as well as 16S rRNA gene copy numbers) and with increased sampling effort (n=20 per treatment). FIG. 10 shows the congruence in the results of this comparison. Tukey's post-hoc tests showed that differences were attributable to the most successful inoculation treatment (cyanobacteria heterotrophs) against controls.

To quantify recovery rates during the second year, we regressed plot Chl a means content against time (FIG. 6). At onset, all treatments had very low biomass (0.6-1.4 mg m⁻² on the order of 1 to 5% of the Chl a level in natural crust patches under PV installations preceding the experiment). Linear rates of recovery under this moderate weather regime ranged from 3.85 to 22.63 mg Chi a m² yr¹, depending on treatment. The rates for the ‘cyanobacteria+heterotrophs’ treatment. The dynamics of recovery based on Chi a are shown in FIG. A. After a moderate pulse of growth following inoculation, a generalized stasis or decline set in, both in controls and treatments, coinciding with the extremely dry and hot spell. The second year provided copious rainfall, and upward trends across controls and treatments were measurable.

By the end of the second year, recovery had occurred in all plots, and differences according to original inoculation treatment were patent FIG. 5 panel A. The best-performing treatment (cyanobacteria+heterotrophs) had reached a Chl a level approaching the summer concentration of natural crusts at the site, although most treatments and the untreated controls fell short of this. To gauge the general effect of rainfall on growth, we quantified the changes in average Chl a biomass attained between samplings (FIG. 5 panel B). We found a moderate trend of increase in relative growth with increasing intervening cumulative rainfall (linear, R²=0.24), but the relationship can be substantially improved if the respective periods were binned into ‘summer’ (15 April to 14 October) and ‘winter’ (15 October to 14 April), with R² of 0.82 (quadratic) and 0.91 (linear), respectively. This reveals that unusually wet monsoons were needed to elicit net growth, whereas much more moderate rainfall in winter had an equivalent effect on growth. Thus, winter rainfall more strongly determined biocrust development and moderate rainfall in summer resulted in biomass loss. This is consistent with previous determinations of biocrust expansion in greenhouse settings²⁶ and with recommendations to avoid Sonoran summertime during biocrust production in nurseries. To assess treatment effects at the end point more accurately, we analyzed biocrust development using several parameters concurrently (Chl a and community DNA concentrations, as well as 16S rRNA gene copy numbers) and with increased sampling effort (n=20 per treatment). FIG. 10 shows the congruence in the results of this com-parison. Tukey's post-hoc tests showed that differences were attributable to the most successful inoculation treatment (cyanobacteria+heterotrophs) against controls.

To quantify recovery rates during the second year, we regressed plot Chi a means content against time (FIG. 6). At onset, all treatments with respect to long-term precipitation patterns. The average monsoon rainfall during the past 25 yr in this area is 57 mm (c.v. 80%), hence the monsoon rainfall in 2020 was indeed extremely dry, whereas in 2021 it was on the high end. For winter rains determinant of biocrust growth, the average is 105 mm (c.v. 60%), hence 2020/2021 was also rather meager, but 2021/2022 was very close to the average. In this context, the level of biocrust recovery attained during the second year constitutes an acceptable long-term estimator.

The average bacterial/archaeal composition of the recovered biocrusts (all types) at the phylum level was indistinguishable from the naturally occurring original crusts. However, the recovered biocrusts were clearly of lower diversity than natural crusts regarding the composition of cyanobacteria, uniformly showing strong dominance by M vaginatus over other biocrust taxa (FIG. 9), much more than found in the original crusts. No major differences were found in cyanobacterial composition between controls and any of the different inocula used, and no cyanobacterial types atypical of the biocrust environment were found under any treatment.

### Discussion

### PV Installations as Biocrust Nurseries

We show that PV installations in solar farms offer a setting naturally conducive to biocrust development in comparison to fully exposed adjacent soil, as judged both by biocrust cover and biocrust areal bio-mass proxies (FIGS. 3 and 10). Cover under the influence of raised PV panels tripled that found in exposed neighbouring soils, and Chl a concentrations almost doubled.

Our control experimental plots quantified recovery of biocrusts by natural processes of microbial colonization (that is, aeolian deposition, overland flow) after complete harvesting. Biocrust recovery was measurable, if moderate: around 5 mg Chl a m⁻² yr⁻¹ (FIG. 6) during the second year. This corresponds to a yearly recovery of some 15% of the long-term steady-state cover on the site. Unaided, without wishing to be bound by theory, full recovery of denuded soil under panels can be achieved in about 6-8 yr.

Recovery rates can accelerated if harvested areas were re-inoculated. In our best-performing treatments (FIG. 5 panel A) and judging from the second year, 1 yr of recovery sufficed to bring biocrust cover close to original levels. This is despite the unexpectedly extreme weather that caused net population losses during the first summer. Surprisingly, we can still measure inoculation effects at the end of the experiment speaks for the resilience of inoculants to extreme weather and their suitability against climatic vagaries. This extremely dry spell can have caused a community bottleneck favouring the most drought-resistant cyanobacterium in North American biocrusts, M. vaginatus^{38,39}, to be selected for in the experimental inoculations. That M. vaginatus is particularly adept at growing under lower temperatures^{36,39} can have exacerbated this differential outcome since most of the recovery can be traced to winter precipitation events (FIG. 5 panel B).

### PV Installations as Sustainable Source of Biocrust Inoculum

Herein, we describe a continuous, low-tech, low-impact operation for solar farms as sustainable sources of biocrust inoculum, in which one can do away with work-intensive, specialized nurseries by simply re-investing part of the biocrust harvest as a boost to natural recovery processes in the areas harvested. Guided by our experience at Poly Ground Mount 2, we describe in FIG. 7 what such a continuous operation can look like: for any given area of PV biocrust harvested, without wishing to be bound by theory, full recovery can occur within 1-2 yr if investing a proportion of the harvest into its re-inoculation and without any nursery production of inoculum or other treatments. We chose 10% as a conservative placeholder for this proportion. This still leaves 90% of the harvest for use in restoration of target degraded soils. If target soils are inoculated at 5% levels, an area 18 times the area harvested can be treated. The original source area can then be harvested recurrently biennially and operated in this mode continuously.

### Assessing the Scalability Promise of Crustivoltaics

We provide herein anon-limiting, illustrative scalability assessment of crustivoltaics for the case of Maricopa County (Arizona, USA). Some 30-40% of its 191,918 ha of farmland⁴² are left fallow because of increasing water scarcity, becoming a problematic source of fugitive dust to the Phoenix Metro area. Its three largest solar farms (Mesquite Solar Project. Solana Generating Station and Arlington Valley II) cover some 1,623 ha (376, 777 and 469, respectively). Their dual use as biocrust nurseries as detailed in the herein can be used to treat 29,214 ha of crust-less soil, which corresponds to some 45% of all of Maricopa County's fallow land. Assuming biocrust regeneration in 2 yr by re-seeding at 10% inoculation, the entirety of Maricopa County fallow lands can be treated within 4-5 yr. Without wishing to be bound by theory, an equipped non-specialist operator can harvest 1,000 m² (0.1 ha) of PV biocrust and sow 1.8 target ha per day, or some 1,188 ha in a 2 yr period. Twenty-five such operators can suffice to conduct the entire operation, which fits within the common definition of a small business. By comparison, in nursery-based technology, one highly trained operator can attain maximally the annual seeding of a few acres, requiring considerable technological investments in equipment, space and monitoring. FIG. 11 provides a quantitative assessment of the actual capacity attained by current biocrust restoration technologies. The crustivoltaics approach, by doing away with the need to produce inoculum in nurseries and relying on existing extensive nursery-like settings, offers cost/return benefits that are at 3-4 orders of magnitude smaller. Crustivoltaics represents a high-capacity, low-cost and resource-efficient new approach.

### a Win-Win Situation

The burden of crustivoltaics on PV farm operations is minimal, requiring only one intervention every 2 yr and no irrigation, certainly much less disruptive than agrivoltaics. It offers the added benefit of promoting net increases in power output by reduction of dust deposition from surrounding crustless areas. Solar farm operators can be interested in partnerships if the prospect of improved energy yields is part of the equation. Through such partnerships, operators can take advantage of incentives for their contribution to sustainability and conservation of natural resources.

One such benefit is claiming carbon sequestration credits, since biocrusts are photosynthetic systems that draw CO₂ from the atmosphere just like plants. In cyanocrusts from warm North American deserts, the ratio of carbon stock to Chl a concentration is 5±3 g C mg¹ (Chl a), calculated from data (n=15) in refs. 8,43, so that an average cyanocrust with 50 mg Chl a m⁻² stocks around 2.5 tons C ha⁻¹ in the top cm of soil. This estimate is consistent with direct measurements of cyanocrust C stocks in China⁴⁴. Repopulating a hectare with biocrusts will thus sequester on average 9 tons of CO₂ from the atmosphere by maturity. For comparison, this is equivalent to the sequestration of 60 pine trees for 20 yr, according to the Winrock International calculator (winrock.org/flr-calculator/) based on ref. 45. At current carbon credit prices of US$32 per ton, monetization of crustivoltaics can reach the order of US$300 ha⁻¹, sufficient to cover a large portion of operating costs.

### Methods

### Site

For our experiment described herein, we selected a small suburban PV installation, Poly Ground Mount 2, in continuous operation by Clearway Energy during the study period and located in Mesa, Arizona (33.3011° N, 111.6829° W), because of ease of access from our Mobile Nursery Facility (some 800 ft. away) and associated continuous climate records. The solar field covers ~24,000 m² and has been in operation since June 2012. The substrate is a coarse-loamy, mixed, calcareous, hyperthermic Typic Torrifluvents soil of the Gilman loam series, roughly 40% sand and 22% clay, slightly alkaline pH (8), containing 12% carbonates and classified as having a wind erodibility index of 86.

### Assessment of Biocrust Development

To determine natural development of biocrusts as a function of PV panel installations, we carried out sampling along four pre-determined linear transects, two under the PV installation and two along the PV panel-free space. Biocrust cover was determined by photography and image analyses in equally spaced 1 m² quadrats⁴². Photographs were taken after a rain event since this reveals existing populations of cyanobacteria that migrate to the surface where present, greening the soil³⁷. Photographs of each quadrant were then analyzed using open-source ImageJ software, by tracing visually green vs non-green patches and calculating areas of each using area determination tools. Determination of % cover in this way is adequate to gauge major differences and integrates cm and dm patchiness effectively, but suffers from low resolution, as microbial populations of low concentration may not be detectable. For this reason, we complemented our surveys with sacrificial sampling to determine Chl a. Chl a is largely a cyanobacteria-specific biomass proxy in the absence of algae and mosses, and is widely used in biocrust research, and can be a measure of cyanobacterial biomass²⁷. In the case of our experimental plots, in addition to Chl a, we complemented our analyses with determinations of total extractable DNA and number of 16S rRNA gene copies per unit area of soil. Total extractable DNA in soils is a proxy for microbial biomass in the absence of plant material and while non-crusted soils also harbour microbial DNA, its concentrations in biocrust are orders of magnitude higher⁵¹. Counts of 16S rRNA are specific for bacterial and archaeal biomass, also used as an alternative to Chl a in biocrust research².

### Biocrust Inoculation

To investigate our ability to promote growth of biocrusts under the PV nursery, we conducted interventional inoculation experiments with various treatments that lasted over 2 yr. For this, a total of 30, 1 m² plots were prepared; 5 per treatment type, placed in a randomized fashion within the area of influence of PV panel rows, avoiding placement in perimetral areas of the facility. We staked the corners of each quadrant with rebar for ease of recognition. Before inoculation, we uprooted a few plants present and scalped the areas of existing biocrust. No grading or perimeter walling was conducted. A spatially explicit pictorial description of the experimental design is shown in FIG. 1.

### Isolation of Local Bacteria for Inoculum Preparation

For the isolation of motile filamentous bundle-forming non-nitrogen fixing cyanobacteria, we wetted remnant biocrusts obtained locally and then picked bundles directly from the soil surface using fine forceps under the dissecting microscope. Bundles were cleaned of soil and attached bacteria by dragging them over 2% (w/v) agar-solidified Jaworski's medium (JM), transferred into 96-well plates containing liquid JM medium, incubated at 25±2° C. and illuminated with 20-30 μmol photon m⁻² s⁻¹ under a 14 h photoperiod²⁴. Once enough biomass was observed, the enrichment was inspected under the microscope and a preliminary taxonomic assignment made on the basis of morphology. Each isolate received a strain identification code, with its identity established by DNA sequencing, and the isolate then cryopreserved until further use. To isolate heterotrophic bacteria that are mutualistic with cyanobacteria for nitrogen fixation and phosphorus solubilization, we used cleaned bundles but incubated in nitrogen-free Burk's⁵⁴ and NBRIY⁵⁵ media, respectively, with added organics (solidified with 1% Gellan gum) and incubated for 20 d (23° C., 18-20 μE m⁻² s⁻¹ of illumination with 14 h light: 10 h dark cycles). Then, individual colonies were picked and streaked on fresh plates, repeating three times for each colony transfer, to obtain pure isolates as previously described in detail (Nelson, C., Microcoleus vaginatus. ISME J. 15, 282-292 (2021)). Seven such isolates were selected for use.

### Inoculum Preparation

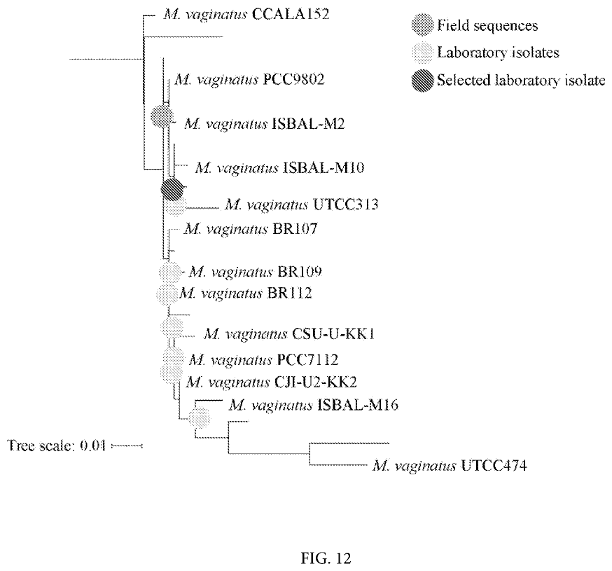

All sources of inoculum were ultimately derived from natural biocrusts present in the area surrounding the facility. We prepared ‘whole community’ inocula by cultivating these biocrusts in the greenhouse of our nursery facility under controlled conditions and rounds of recurrent irrigation/desiccation with a FISS system²⁷, over a 1 cm deep substrate of local soil. The resulting artificial biocrusts were subjected to molecular quality control, then collected when they reached a Chl a concentration of 80 mg m⁻² and crumbled into mm-large pieces for manual sowing over the plot. Cyanobacterial culture inoculum was prepared as follows: two pedigreed cyanobacterial strains matched genetically to those present in the natural crusts (FIG. 12) were grown under sterile conditions in liquid JM medium under laboratory conditions, and cultures scaled up to the desired yield as explained in detail elsewhere²⁴. These cultures were then mixed with local soil and incubated once more as a mixture of culture/soil under more natural conditions in the greenhouse using the FISS technology, to obtain enough biomass while ‘hardening’ the cultures. They were collected at a concentration of 13 mg Chl a m⁻², in preparation for field inoculation. After quality control, they were dried and crumbled and mm-sized pieces were then sown onto the experimental quadrants. Heterotrophic mutualistic isolates were grown in the laboratory in large liquid cultures, harvested and mixed into a cocktail in equal proportions to an optical density (OD)₆₀₀ of 0.1. Of the cocktail, 200 ml was applied per m² of crusts directly on the experimental plots.

### Chl a Content

Chl a was determined spectrophotometrically after 90% acetone extraction of soil ground with a mortar and pestle for 3 min²⁴. At least five samples of the top cm of the soil were taken randomly, covering a total area of at least 6.8 cm², and mixed into a slurry. Slurries were placed in test tubes, the volume adjusted to 10 ml with 90% acetone and chlorophyll extracted for 24 h at 4° C. for a single consolidated determination. Extract absorbance spectra were recorded on a Shimadzu UV-1601 spectrophotometer. Interference from scytonemin and carotenoids was discounted using trichromatic equations².

### DNA Extraction and Molecular Methodology

Genomic DNA was extracted from 0.25 g of soil using the Powersoil Pro extraction kit (QIAGEN), following manufacturer instructions. Three of these extracts were mixed and the mixture used for quantification of total DNA and number of 16S rRNA copies. Determination of microbial community composition was performed on three independent extracts. Total DNA was quantified using a Quant-iT PicoGreen dsDNA Assay kit (Invitrogen). For 16S rRNA gene quantitation, a universal (bac-terial/archaeal) primer set (338F 5′-ACTCCTACGG GAGGCAGCAG-3′, 518R 5′-GTATTACCG CGGCTGCTGG-3)⁵⁷ was used. The qPCR reactions were performed in triplicate using PerfeCTa SYBR Green FastMix Rox (Quantabio) in an ABI ViiA 7 thermocycler (Applied Biosystems). Micro-bial community structure was determined through next generation sequencing of 16S rRNA genes. General bacterial primers 515F/806R were used to amplify the V4 region of 16S rRNA genes, according to the Earth Microbiome Project⁵⁸. PCR was performed in triplicate and library was prepared to a final concentration of 4 pM. PhiX (180 μl, Illumina) at a concentration of 12 pM and 150 μl of buffer HT1 (Illumina) were mixed with 270 μl of the pooled library and loaded in the MiSeq Illumina sequencer, adding custom 16S rRNA sequencing primers on a paired-ends sequencing flow cell 2 (2×250 bp). Sequencing yielded raw FASTQ sequence files.

### Bioinformatic Analyses

Paired-end reads obtained from Illumina sequencing were demultiplexed, and feature tables containing representative sequences and their frequencies were obtained using the DADA2 plugin⁵⁹ available in Qiime2 v2020.11⁶⁰. The resulting feature table was filtered out to remove rare features. Highly variable positions were removed using MAFFT⁶¹, and phylogenetic trees were generated using FastTree⁶². Preliminary taxonomic assignments were done with the Ribosomal Database Project classifier⁶³, and representative sequences were then aligned against the Greengenes 13_8 database core reference align-ment⁶⁴. Cyanobacterial and non-cyanobacterial sequences were then split into separate feature tables⁶⁵ and the cyanobacterial features were further curated via Cydrasil3⁶⁶ (www.cvdrasil.org).

### Statistics

We used t-test to assess the significance of measured parameters between two groups and one-way analysis of variance (ANOVA) to test differences among treatments. These tests were run in Excel. We found that data for all biomass parameters were not normally distributed according to Shapiro-Wilk test (using the R package stats⁶⁷). While in principle one can still confidently use t-test and ANOVA because the number of observations was high (>20), we also concurrently ran the non-parametric Kruskal-Wallis test using R⁶⁷, and report significance for both parametric and non-parametric tests. To test for specific treatment drivers of significance in ANOVA, we used Tukey's multiple comparisons of means in R⁶⁷. For % cover, data were transformed using the angular transformation for percentages before testing. An analysis of covariance (ANCOVA) was carried out using R⁶⁷ to compare the rates of biocrust recovery during the 2021/2022 season according to initial inoculation treatment. Homogeneity of variance was tested using Levene's test in the R package Car⁶⁸.

### Data Availability

Molecular sequence data for strain and microbial community analyses are available from NCBI under bioproject number PRJNA899057. Context databases used for phylogenetic placement are available at github.com/anagiraldo/Crustivoltaics. Other source data used in graphical displays or statistical analyses are provided as supplementary files. Weather records used for context are available at alert.fcd.maricopa.gov/alert/Google/v3/gmap.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A biocrust bacteria inoculum, the inoculum comprising cyanobacteria, heterotrophic bacteria, or a combination thereof, wherein the inoculum inoculates a soil to generate a biocrust.

2. The inoculum of claim 1, wherein the cyanobacteria comprise bacteria of the Microcoleaceae family or Coleofasciculaceae family.

3. The inoculum of claim 2, wherein the bacteria of the Microcoleaceae family comprise Microcoleus vaginatus, Microcoleus vaginatus BN15, or a combination thereof.

4. The inoculum of claim 2, wherein the bacteria of the Coleofasciculaceae family comprise Pycnacronema, Crassifilum, Crassifilum solicrustae BN2, or a combination thereof.

5. The inoculum of claim 1, wherein the heterotrophic bacteria comprise mutualistic isolates.

6. A method of developing large biocrust nurseries, the method comprising:
isolating bacteria from a remnant biocrust;
performing a taxonomic assignment based on morphology of the isolated bacteria;
generating an inoculum comprising bacteria similar in community composition and/or genetic identity to bacteria identified in the remnant biocrust; and
inoculating a recipient soil under a photovoltaic (PV) installation to generate a biocrust in a target soil.

7. The method of claim 6, further comprising measuring a microbial parameter in a target soil.

8. The method of claim 7, wherein the microbial parameter comprises a visual survey, Chlorophyll a (Chl a) concentration, total extractable DNA concentration, number of 16S rRNA gene copies per unit area of soil, or combinations thereof.

9. The method of claim 6, wherein the method further comprises inoculating a second target soil with the first target soil of claim 6.

10. The method of claim 9, wherein the second target soil is in a natural setting or an artificial setting.

11. The method of claim 9, wherein the second target soil inoculates a third target soil.

12. The method of claim 9, wherein the inoculation of subsequent target soil(s) by the second or third target soils comprise a continuous process.

13. A biocrust nursery system comprising:
a photovoltaic (PV) energy installation;
the biocrust inoculum of claim 1; and
a target soil, wherein the target soil is located beneath the PV energy installation.

14. The biocrust nursery system of claim 13, wherein the system functions as both a solar power plant and as a biocrust nursery.

15. The biocrust nursery system of claim 14, wherein the target soil inoculates a second target soil creating a cycling nursery system.

16. The biocrust nursery system of claim 15, wherein the system is continuous.

17.-24. (canceled)
